(19) United States
(12) Patent Application Publication
(10) Pub. No.: US 2020/0131177 A1
(43) Pub. Date: Apr. 30, 2020

(54) PESTICIDALLY ACTIVE HETEROCYCLIC DERIVATIVES WITH SULFUR CONTAINING SUBSTITUENTS

(71) Applicant: SYNGENTA PARTICIPATIONS AG, Basel (CH)

(72) Inventors: Sebastian RENDLER, Stein (CH); Andrew EDMUNDS, Stein (CH); Michel MUEHLEBACH, Stein (CH); Daniel EMERY, Stein (CH); Girish RAWAL, Goa (IN); Indira SEN, Goa (IN); Vikas SIKERVAR, Goa (IN)

(73) Assignee: SYNGENTA PARTICIPATIONS AG, Basel (CH)

(21) Appl. No.: 16/629,017

(22) PCT Filed: Jul. 5, 2018
(86) PCT No.: PCT/EP2018/068296

§ 371 Date: Jan. 6, 2020

(30) Foreign Application Priority Data
Jul. 7, 2017 (IN) 201711024063

(51) Int. Cl. C07D 471/04; A01N 43/90

(52) U.S. Cl. CPC C07D 471/04; A01N 43/90

(57) ABSTRACT

Compounds of formula (I), wherein the substituents are as defined in claim 1, and the agrochemically acceptable salts salts, stereoisomers, enantiomers, tautomers and N-oxides of those compounds, can be used as insecticides and can be prepared in a manner known per se.

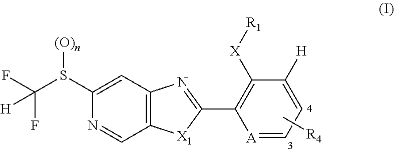

The present invention relates to pesticidally active, in particular insecticidally active heterocyclic derivatives containing sulfur substituents, to intermediates for the preparation of those compounds, to compositions comprising those compounds, and to their use for controlling animal pests (including arthropods and in particular insects or representatives of the order Acarina).

Heterocyclic compounds with pesticidal action are known and described, for example, in WO 2015/000715, WO 2015/121136, WO 2016/026848, WO 2016/030229, WO 2016/107831 and WO2017/082132.

There have now been found novel pesticidally active heterocyclic 6/5-bicyclic ring derivatives with sulfur containing phenyl and pyridyl groups which are characterized by a difluoromethylthio, difluoromethylsulfinyl or difluoromethylsulfonyl group attached to the bicyclic ring.

The present invention accordingly relates to compounds of formula I,

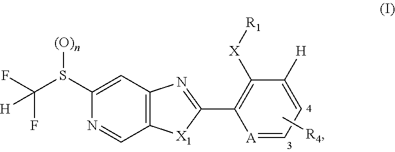

wherein

### A is CH or N;

### X is S, SO or SO₂;

R₁ is C₁-C₄alkyl, C₁-C₄haloalkyl or C₃-C₆cycloalkylC₁-C₄alkyl;
n is 0, 1 or 2;
X₁ is O, S or NR³, wherein R³ is C₁-C₄alkyl; and
R₄ is hydrogen, halogen, C₁-C₆alkyl, C₁-C₆haloalkyl, C₁-C₆haloalkoxy, C₃-C₆cycloalkyl, C₃-C₆cycloalkyl-C₁-C₄alkyl, C₁-C₆cyanoalkyl, C₁-C₆alkoxy, C₁-C₆cyanoalkoxy, C₃-C₆cycloalkyl-C₁-C₄alkoxy or C₃-C₆halocycloalkyl-C₁-C₄alkoxy; or
R₄ is C₃-C₆cycloalkyl mono - or polysubstituted by substituents selected from the group consisting of halogen, cyano, C₁-C₄alkyl, C₁-C₄haloalkyl and C₃-C₆cycloalkyl; or
R₄ is a five- to ten-membered monocyclic or fused bicyclic heterocyclic ring system which can be aromatic, partially saturated or fully saturated and which contains 1 to 4 hetero atoms selected from the group consisting of nitrogen, oxygen and sulfur, with the proviso that each ring system cannot contain more than 2 oxygen atoms and not more than 2 sulfur atoms, said five- to ten-membered ring system can be mono- to polysubstituted by substituents independently selected from the group consisting of halogen, cyano, oxo, OH, NH₂, C₁-C₆alkyl, C₃-C₆cycloalkyl, C₁-C₆haloalkyl, C₁-C₆alkoxy, C₁-C₆alkylthio, and C₁-C₆alkylsulfonyl; and agrochemically acceptable salts, stereoisomers, enantiomers, tautomers and N-oxides of the compounds of formula I.

Compounds of formula I which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula I which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

The alkyl groups occurring in the definitions of the substituents can be straight-chain or branched and are, for example, methyl, ethyl, n-propyl, isopropyl, n-butyl, sec-butyl, iso-butyl, tert-butyl, pentyl, hexyl, and their branched isomers. Alkylthio, alkylsulfonyl, alkylsulfinyl, and alkoxy radicals are derived from the alkyl radicals mentioned.

Alkylsulfanyl is for example methylsulfanyl, ethylsulfanyl, propylsulfanyl, isopropylsulfanyl, butylsulfanyl, pentylsulfanyl, and hexylsulfanyl.

Alkylsulfinyl is for example methylsulfinyl, ethylsulfinyl, propylsulfinyl, isopropylsulfinyl, butylsulfinyl, pentylsulfinyl, and hexylsulfinyl.

Alkylsulfonyl is for example methylsulfonyl, ethylsulfonyl, propylsulfonyl, isopropylsulfonyl, butylsulfonyl, pentylsulfonyl, and hexylsulfonyl.

Halogen is generally fluorine, chlorine, bromine or iodine. This also applies, correspondingly, to halogen in combination with other meanings, such as haloalkyl.

Haloalkyl groups preferably have a chain length of from 1 to 6 carbon atoms. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl.

Alkoxy groups preferably have a preferred chain length of from 1 to 6 carbon atoms. Alkoxy is, for example, methoxy, ethoxy, propoxy, i-propoxy, n-butoxy, isobutoxy, sec-butoxy and tert-butoxy and also the isomeric pentyloxy and hexyloxy radicals.

Alkoxyalkyl groups preferably have a chain length of 1 to 6 carbon atoms.

Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

The cycloalkyl groups preferably have from 3 to 6 ring carbon atoms, for example cyclopropyl, cyclobutyl, cyclopentyl and cyclohexyl.

Haloalkoxy groups preferably have a chain length of from 1 to 4 carbon atoms. Haloalkoxy is, for example, difluoromethoxy, trifluoromethoxy or 2,2,2-trifluoroethoxy.

Free radicals represent methyl groups.

In the heterocycles J, which represent embodiments of the substituent R4, the free radical represents a bond.

The compounds of formula I according to the invention also include hydrates which may be formed during the salt formation.

According to the present invention, five- to ten-membered monocyclic or fused bicyclic ring system which can be aromatic, partially saturated or fully saturated and contains 1 to 4 hetero atoms selected from the group consisting of nitrogen, oxygen and sulfur, it not being possible for each ring system to contain more than 2 oxygen atoms and more than 2 sulfur atoms or a three- to ten-membered, monocyclic or fused bicyclic ring system which may be aromatic, partially saturated or fully saturated; is, depending of the number of ring members, for example, selected from the group consisting of the following heterocyclic groups:

pyrrolyl; pyrazolyl; isoxazolyl; furanyl; thienyl; imidazolyl; oxazolyl; thiazolyl; isothiazolyl; triazolyl; oxadiazolyl; thiadiazolyl; tetrazolyl; furyl; pyridyl; pyrimidyl; pyrazinyl; pyridazinyl; triazinyl, pyranyl; quinazolinyl; isoquinolinyl; indolizinyl; isobenzofuranylnaphthyridinyl; quinoxalinyl; cinnolinyl; phthalazinyl; benzothiazolyl; benzoxazolyl; benzotriazolyl; indazolyl; indolyl; pyrrolidinyl; piperidinyl; morpholinyl; indolinyl; tetrahydroquinolinyl;

### (1H-pyrrol-1-yl)-; (1H-pyrrol-2-yl)-; (1H-pyrrol-3-yl)-;

### (1H-pyrazol-1-yl)-; (1H-pyrazol-3-yl)-; (3H-pyrazol-3-yl)-; (1H-pyrazol-4-yl)-;

(3-isoxazolyl)-; (5-isoxazolyl)-;
(2-furanyl)-; (3-furanyl)-;
(2-thienyl)-; (3-thienyl)-;

### (1H-imidazol-2-yl)-; (1H-imidazol-4-yl)-; (1H-imidazol-5-yl)-;

(2-oxazol-2-yl)-; (oxazol-4-yl)-; (oxazol-5-yl)-;
(thiazol-2-yl)-; (thiazol-4-yl)-; (thiazol-5-yl)-;
(isothiazol-3-yl)-; (isothiazol-5-yl)-;
(1H-1,2,3-triazol-1-yl)-; (1H-1,2,4-triazol-3-yl)-; (4H-1,2,4-triazol-4-yl)-; (1H-1,2,4-triazol-1-yl)-(1,2,3-oxadiazol-2-yl)-; (1,2,4-oxadiazol-3-yl)-; (1,2,4-oxadiazol-4-yl)-; (1,2,4-oxadiazol-5-yl)-;
(1,2,3-thiadiazol-2-yl)-; (1,2,4-thiadiazol-3-yl)-; (1,2,4-thiadiazol-4-yl)-; (1,3,4-thiadiazol-5-yl)-;

### (1H-tetrazol-1-yl)-; (1H-tetrazol-5-yl)-; (2H-tetrazol-5-yl)-;

(2-pyridyl)-; (3-pyridyl)-; (4-pyridyl)-;
(2-pyrimidinyl)-; (4-pyrimidinyl)-; (5-pyrimidinyl)-;
(2-pyrazinyl)-;
(3-pyridazinyl)-; (4-pyridazinyl)-;
(1,3,5-triazin-2-yl)-; (1,2,4-triazin-5-yl)-; (1,2,4-triazin-6-yl)-; (1,2,4-triazin-3-yl)-;
(furazan-3-yl)-;
(2-quinolinyl)-; (3-quinolinyl)-; (4-quinolinyl)-; (5-quinolinyl)-; (6-quinolinyl)-; (3-isoquinolnyl)-; (4-isoquinolnyl)-; (2-quinozolinyl)-; (2-quinoxalinyl)-; (5-quinoxalinyl)-; (pyrido[2,3-b]pyrazin-7-yl)-;
(benzoxazol-5-yl)-; (benzothiazol-5-yl)-; (benzo[b]thien-2-yl) and (benzo[1,2,5]oxadiazol-5-yl).

In a preferred embodiment of the invention,

R₄ is a five- to ten-membered monocyclic or fused bicyclic heterocyclic ring system which can be aromatic, partially saturated or fully saturated and which contains 1 to 4 hetero atoms selected from the group consisting of nitrogen, oxygen and sulfur, with the proviso that each ring system cannot contain more than 2 oxygen atoms and not more than 2 sulfur atoms, said five- to ten-membered ring system can be mono- to polysubstituted by substituents independently selected from the group consisting of halogen, cyano, oxo, OH, NH₂, C₁-C₆alkyl, C₃-C₆cycloalkyl, C₁-C₆haloalkyl, C₁-C₆alkoxy, C₁-C₆alkylthio, and C₁-C₆alkylsulfonyl, pyrimidinyl, thiazolyl, pyridinyl, pyrazinyl, pyridazinyl, pyrazinyloxy, imidazolyl, dihydro-oxazolyl, oxazolyl, dioxolanyl, 1,3-dioxanyl and 5,6-dihydro-[1,4,2]dioxazinyl; wherein said pyrimidinyl, thiazolyl, pyridinyl, pyrazinyl, pyridazinyl, pyrazinyloxy, imidazolyl, dihydro-oxazolyl, oxazolyl, dioxolanyl, 1,3-dioxanyl and 5,6-dihydro-[1,4,2]dioxazinyl which itself can be mono- to polysubstituted by substituents independently selected from the group consisting of halogen, cyano, oxo, OH, NH₂, C₁-C₆alkyl, C₃-C₆cycloalkyl, C₁-C₆haloalkyl, C₁-C₆alkoxy, C₁-C₆alkylthio and C₁-C₆alkylsulfonyl.

In preferred compounds of formula I, R₄ is selected from the group consisting of J-1 to J-56

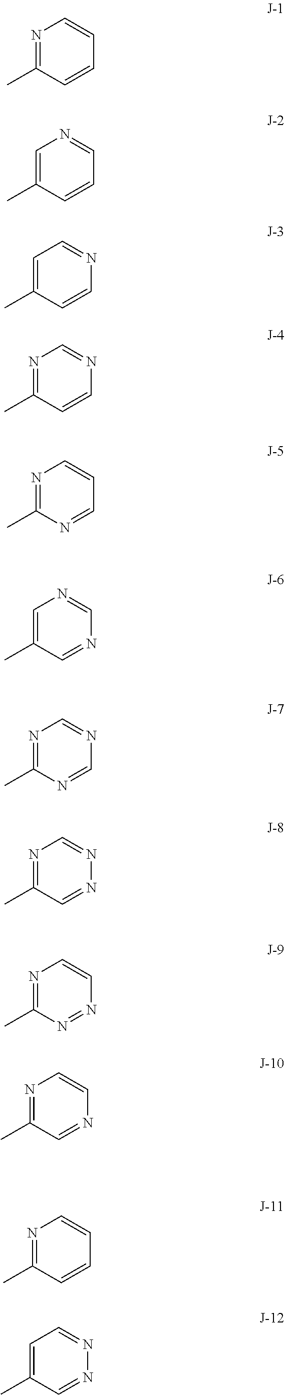

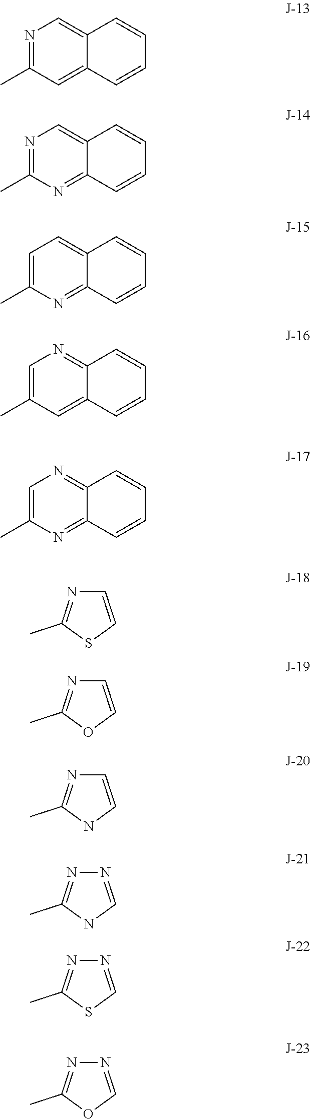

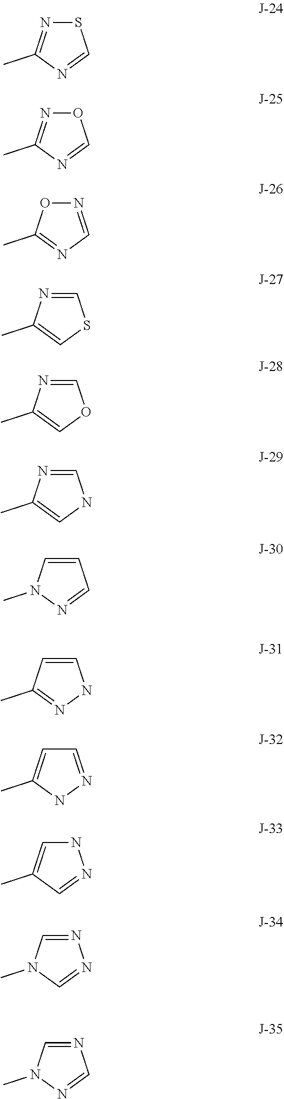

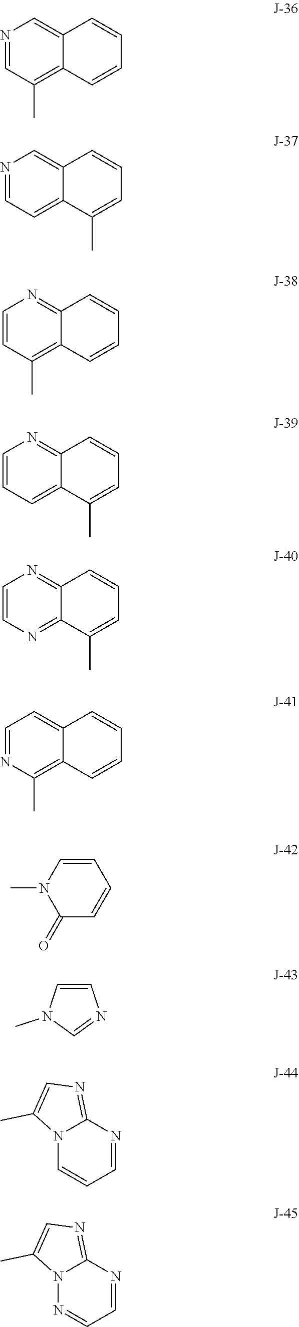

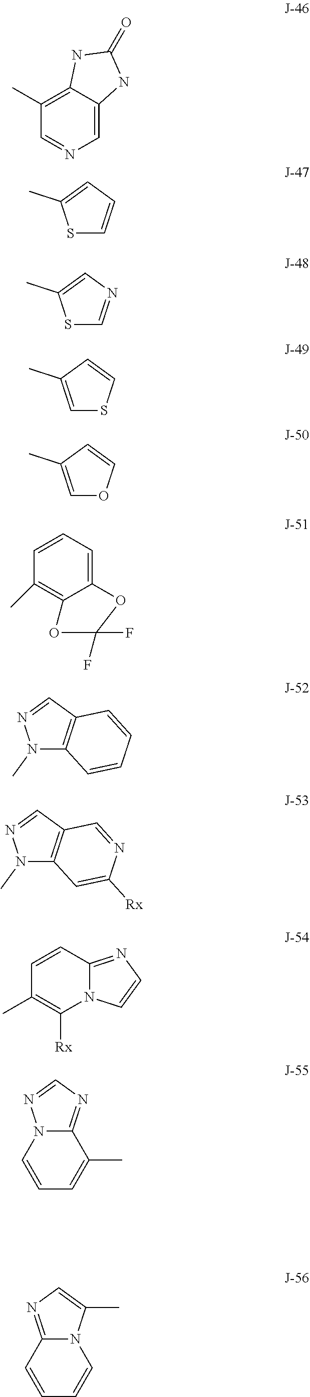

in particular selected from J-1 to J-40;
wherein each group J-1 to J-56 is mono- di- or trisubstituted with Rx, wherein
each Rx is, independently selected from the group consisting of halogen, cyano, oxo, OH, NH₂, C₁-C₆alkyl, C₃-C₆cycloalkyl, C₁-C₆haloalkyl, C₁-C₆alkoxy, C₁-C₆alkylthio and C₁-C₆alkylsulfonyl.

In the context of this invention “mono- to polysubstituted” in the definition of the substituents, means typically, depending on the chemical structure of the substituents, monosubstituted to seven-times substituted, preferably monosubstituted to five-times substituted, more preferably mono-, double- or triple-substituted.

A preferred group of compounds of formula I is represented by the compounds of formula I-1

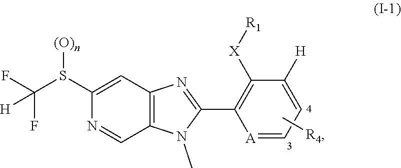

wherein A, X, R₁, R₄, and n are as defined under formula I above.

In said preferred group of compounds of formula I-1,

R₁ is preferably methyl, ethyl, n-propyl, i-propyl or cyclopropylmethyl; and
R₄ is preferably hydrogen, halogen, C₁-C₆alkyl, C₁-C₆haloalkyl, C₁-C₆haloalkoxy, C₃-C₆cycloalkyl, C₃-C₆cycloalkyl-C₁-C₄alkyl, C₁-C₆cyanoalkyl, C₁-C₆alkoxy, C₁-C₆cyanoalkoxy, C₃-C₆cycloalkyl-C₁-C₄alkoxy or C₃-C₆halocycloalkyl-C₁-C₄alkoxy; or C₃-C₆cycloalkyl mono - or polysubstituted by substituents selected from the group consisting of halogen, cyano, C₁-C₄alkyl, C₁-C₄haloalkyl and C₃-C₆cycloalkyl; or is 1,2,4-triazol-1-yl, pyrazol-1-yl, 4-chloropyrazol-1-yl, 4-fluoropyrazol-1-yl or 3-chloropyrazol-1-yl.

Especially preferred compounds of formula I-1 are those, wherein R₁ is ethyl and X is SO₂.

A more preferred group of compounds of formula I-1 is represented by the compounds of formula I-1a

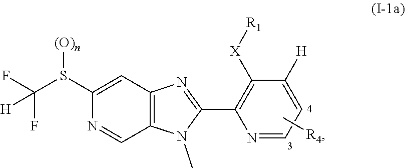

wherein X, R₁, R₄, and n are as defined under formula I above.

In said preferred group of compounds of formula I-1a,

R₁ is preferably methyl, ethyl, n-propyl, i-propyl or cyclopropylmethyl; and
R₄ is preferably hydrogen, halogen, C₁-C₆alkyl, C₁-C₆haloalkyl, C₁-C₆haloalkoxy, C₃-C₆cycloalkyl, C₃-C₆cycloalkyl-C₁-C₄alkyl, C₁-C₆cyanoalkyl, C₁-C₆alkoxy, C₁-C₆cyanoalkoxy, C₃-C₆cycloalkyl-C₁-C₄alkoxy or C₃-C₆halocycloalkyl-C₁-C₄alkoxy; or C₃-C₆cycloalkyl mono - or polysubstituted by substituents selected from the group consisting of halogen, cyano, C₁-C₄alkyl, C₁-C₄haloalkyl and C₃-C₆cycloalkyl; or is 1,2,4-triazol-1-yl, pyrazol-1-yl, 4-chloropyrazol-1-yl, 4-fluoropyrazol-1-yl or 3-chloropyrazol-1-yl.

Especially preferred compounds of formula I-1a are those, wherein R₁ is ethyl and X is SO₂.

Another more preferred group of compounds of formula I-1 is represented by the compounds of formula I-1 b

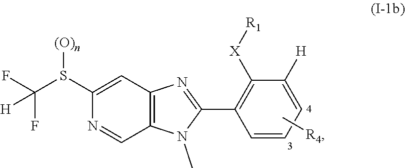

wherein X, R₁, R₄, and n are as defined under formula I above.

In said preferred group of compounds of formula I-1b,

R₁ is preferably methyl, ethyl, n-propyl, i-propyl or cyclopropylmethyl; and
R₄ is preferably hydrogen, halogen, C₁-C₆alkyl, C₁-C₆haloalkyl, C₁-C₆haloalkoxy, C₃-C₆cycloalkyl, C₃-C₆cycloalkyl-C₁-C₄alkyl, C₁-C₆cyanoalkyl, C₁-C₆alkoxy, C₁-C₆cyanoalkoxy, C₃-C₆cycloalkyl-C₁-C₄alkoxy or C₃-C₆halocycloalkyl-C₁-C₄alkoxy; or C₃-C₆cycloalkyl mono - or polysubstituted by substituents selected from the group consisting of halogen, cyano, C₁-C₄alkyl, C₁-C₄haloalkyl and C₃-C₆cycloalkyl; or is 1,2,4-triazol-1-yl, pyrazol-1-yl, 4-chloropyrazol-1-yl, 4-fluoropyrazol-1-yl or 3-chloropyrazol-1-yl.

Especially preferred compounds of formula I-1 b are those, wherein R₁ is ethyl and X is SO₂.

A particularly preferred group of compounds of formula I is represented by the compounds of formula I-2

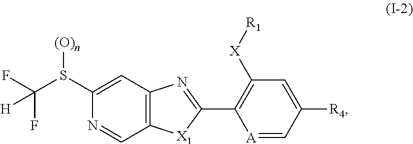

wherein A, X, X₁, R₁, R₄ and n are as defined under formula I above.

In said preferred group of compounds of formula I-2,

R₁ is preferably methyl, ethyl, n-propyl, i-propyl or cyclopropylmethyl; and
R₄ is preferably hydrogen, halogen, C₁-C₆alkyl, C₁-C₆haloalkyl, C₁-C₆haloalkoxy, C₃-C₆cycloalkyl, C₃-C₆cycloalkyl-C₁-C₄alkyl, C₁-C₆cyanoalkyl, C₁-C₆alkoxy, C₁-C₆cyanoalkoxy, C₃-C₆cycloalkyl-C₁-C₄alkoxy or C₃-C₆halocycloalkyl-C₁-C₄alkoxy; or C₃-C₆cycloalkyl mono - or polysubstituted by substituents selected from the group consisting of halogen, cyano, C₁-C₄alkyl, C₁-C₄haloalkyl and C₃-C₆cycloalkyl; or is 1,2,4-triazol-1-yl, pyrazol-1-yl, 4-chloropyrazol-1-yl, 4-fluoropyrazol-1-yl or 3-chloropyrazol-1-yl.

Especially preferred compounds of formula I-2 are those, wherein R₁ is ethyl and X is SO₂.

A more particularly preferred group of compounds of formula I-2 is represented by the compounds of formula I-2a

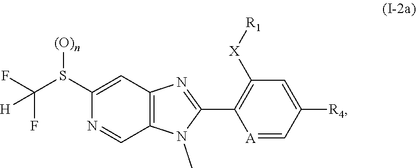

wherein A, X, R₁, R₄ and n are as defined under formula I above.

In said preferred group of compounds of formula I-2a,

R₁ is preferably methyl, ethyl, n-propyl, i-propyl or cyclopropylmethyl; and
R₄ is preferably hydrogen, halogen, C₁-C₆alkyl, C₁-C₆haloalkyl, C₁-C₆haloalkoxy, C₃-C₆cycloalkyl, C₃-C₆cycloalkyl-C₁-C₄alkyl, C₁-C₆cyanoalkyl, C₁-C₆alkoxy, C₁-C₆cyanoalkoxy, C₃-C₆cycloalkyl-C₁-C₄alkoxy or C₃-C₆halocycloalkyl-C₁-C₄alkoxy; or C₃-C₆cycloalkyl mono - or polysubstituted by substituents selected from the group consisting of halogen, cyano, C₁-C₄alkyl, C₁-C₄haloalkyl and C₃-C₆cycloalkyl; or is 1,2,4-triazol-1-yl, pyrazol-1-yl, 4-chloropyrazol-1-yl, 4-fluoropyrazol-1-yl or 3-chloropyrazol-1-yl.

Especially preferred compounds of formula I-2a are those, wherein R₁ is ethyl and X is SO₂.

An even more particularly preferred group of compounds of formula I-2a is represented by the compounds of formula I-2b

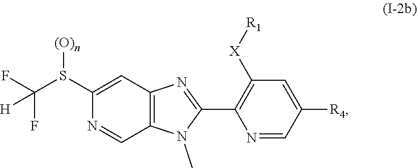

wherein X, R₁, R₄ and n are as defined under formula I above.

In said preferred group of compounds of formula I-2b,

R₁ is preferably methyl, ethyl, n-propyl, i-propyl or cyclopropylmethyl; and
R₄ is preferably hydrogen, halogen, C₁-C₆alkyl, C₁-C₆haloalkyl, C₁-C₆haloalkoxy, C₃-C₆cycloalkyl, C₃-C₆cycloalkyl-C₁-C₄alkyl, C₁-C₆cyanoalkyl, C₁-C₆alkoxy, C₁-C₆cyanoalkoxy, C₃-C₆cycloalkyl-C₁-C₄alkoxy or C₃-C₆halocycloalkyl-C₁-C₄alkoxy; or C₃-C₆cycloalkyl mono - or polysubstituted by substituents selected from the group consisting of halogen, cyano, C₁-C₄alkyl, C₁-C₄haloalkyl and C₃-C₆cycloalkyl; or is 1,2,4-triazol-1-yl, pyrazol-1-yl, 4-chloropyrazol-1-yl, 4-fluoropyrazol-1-yl or 3-chloropyrazol-1-yl.

Especially preferred compounds of formula I-2b are those, wherein R₁ is ethyl and X is SO₂.

Another even more particularly preferred group of compounds of formula I-2a is represented by the compounds of formula I-2c

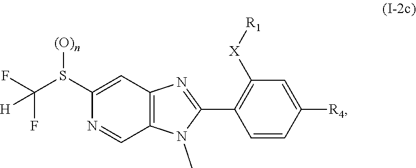

wherein X, R₁, R₄ and n are as defined under formula I above.

In said preferred group of compounds of formula I-2c,

R₁ is preferably methyl, ethyl, n-propyl, i-propyl or cyclopropylmethyl; and
R₄ is preferably hydrogen, halogen, C₁-C₆alkyl, C₁-C₆haloalkyl, C₁-C₆haloalkoxy, C₃-C₆cycloalkyl, C₃-C₆cycloalkyl-C₁-C₄alkyl, C₁-C₆cyanoalkyl, C₁-C₆alkoxy, C₁-C₆cyanoalkoxy, C₃-C₆cycloalkyl-C₁-C₄alkoxy or C₃-C₆halocycloalkyl-C₁-C₄alkoxy; or C₃-C₆cycloalkyl mono - or polysubstituted by substituents selected from the group consisting of halogen, cyano, C₁-C₄alkyl, C₁-C₄haloalkyl and C₃-C₆cycloalkyl; or is 1,2,4-triazol-1-yl, pyrazol-1-yl, 4-chloropyrazol-1-yl, 4-fluoropyrazol-1-yl or 3-chloropyrazol-1-yl.

Especially preferred compounds of formula I-2c are those, wherein R₁ is ethyl and X is SO₂.

Another particularly preferred group of compounds of formula I is represented by the compounds of formula I-3

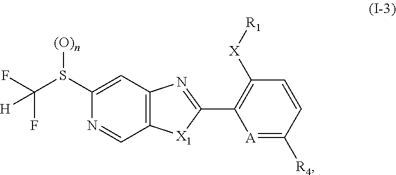

wherein A, X, X₁, R₁, R₄ and n are as defined under formula I above.

In said preferred group of compounds of formula I-3,

R₁ is preferably methyl, ethyl, n-propyl, i-propyl or cyclopropylmethyl; and
R₄ is preferably hydrogen, C₁-C₆alkyl, halogen, C₁-C₆haloalkyl, 1,2,4-triazol-1-yl, pyrazol-1-yl, 4-chloropyrazol-1-yl, 4-fluoropyrazol-1-yl, 3-chloropyrazol-1-yl or pyrimidin-2-yl.

Especially preferred compounds of formula I-3 are those, wherein R₁ is ethyl and X is SO₂.

A more particularly preferred group of compounds of formula I-3 is represented by the compounds of formula I-3a

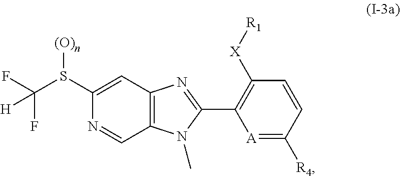

wherein A, X, R₁, R₄ and n are as defined under formula I above.

In said preferred group of compounds of formula I-3a,

R₁ is preferably methyl, ethyl, n-propyl, i-propyl or cyclopropylmethyl; and
R₄ is preferably hydrogen, C₁-C₆alkyl, halogen, C₁-C₆haloalkyl, 1,2,4-triazol-1-yl, pyrazol-1-yl, 4-chloropyrazol-1-yl, 4-fluoropyrazol-1-yl, 3-chloropyrazol-1-yl or pyrimidin-2-yl.

Especially preferred compounds of formula I-3a are those, wherein R₁ is ethyl and X is SO₂.

An even more particularly preferred group of compounds of formula I-3a is represented by the compounds of formula I-3b

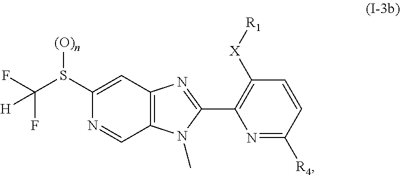

wherein X, R₁, R₄ and n are as defined under formula I above.

In said preferred group of compounds of formula I-3b,

R₁ is preferably methyl, ethyl, n-propyl, i-propyl or cyclopropylmethyl; and
R₄ is preferably hydrogen, C₁-C₆alkyl, halogen, C₁-C₆haloalkyl, 1,2,4-triazol-1-yl, pyrazol-1-yl, 4-chloropyrazol-1-yl, 4-fluoropyrazol-1-yl, 3-chloropyrazol-1-yl or pyrimidin-2-yl.

Especially preferred compounds of formula I-3b are those, wherein R₁ is ethyl and X is SO₂.

An even more particularly preferred group of compounds of formula I-3a is represented by the compounds of formula I-3c

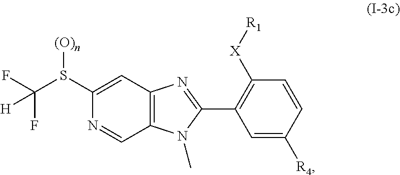

wherein X, R₁, R₄ and n are as defined under formula I above.

In said preferred group of compounds of formula I-3b,

R₁ is preferably methyl, ethyl, n-propyl, i-propyl or cyclopropylmethyl; and
R₄ is preferably hydrogen, C₁-C₆alkyl, halogen, C₁-C₆haloalkyl, 1,2,4-triazol-1-yl, pyrazol-1-yl, 4-chloropyrazol-1-yl, 4-fluoropyrazol-1-yl, 3-chloropyrazol-1-yl or pyrimidin-2-yl.

Especially preferred compounds of formula I-3c are those, wherein R₁ is ethyl and X is SO₂.

In all of the preferred embodiments mentioned above, most preferably

### X is SO₂.

In all of the preferred embodiments mentioned above, most preferably

R₁ is ethyl.

In all of the preferred embodiments mentioned above, most preferably

X₁═NR³, wherein R³ is methyl.

In all of the preferred embodiments mentioned above, most preferably

R₄ is hydrogen, C₁-C₆haloalkyl, 1,2,4-triazol-1-yl, pyrazol-1-yl, 4-chloropyrazol-1-yl, 4-fluoropyrazol-1-yl or 3-chloropyrazol-1-yl.

Particularly preferred are compounds of formula I, wherein

R₁ is C₁-C₄alkyl, preferably ethyl;
X₁═NR³, wherein R³ is methyl;

### A is CH or N;

X is S or SO₂; preferably SO₂; and
R₄ is hydrogen, C₁-C₆haloalkyl, C₁-C₆haloalkoxy, C₁-C₆alkoxy, C₁-C₆cyanoalkoxy, C₃-C₆cycloalkyl-C₁-C₄alkoxy, 1,2,4-triazol-1-yl, pyrazol-1-yl, 4-chloropyrazol-1-yl, 4-fluoropyrazol-1-yl, 3-chloropyrazol-1-yl; preferably hydrogen, trifluoromethyl, ispropoxy or 1,2,4-triazol-1-yl.

The process according to the invention for preparing compounds of formula I is carried out by methods known to those skilled in the art.

Compounds of formula IC, wherein n=2, X is SO₂, A, X₁, R₁ and R₄ are defined as under formula I above, may be prepared by oxidation of compounds of formula IB, wherein n=1, X is SO₂, A, X₁, R₁ and R₄ are defined as under formula I above. The reaction can be performed with reagents such as a peracid, for example peracetic acid or m-chloroperbenzoic acid, or a hydroperoxide, as for example, hydrogen peroxide or tert-butylhydroperoxide, or an inorganic oxidant, such as a monoperoxo-disulfate salt or potassium permanganate.

In a similar way, compounds of formula IB, wherein n=1, X is SO₂, A, X₁, R₁ and R₄ are defined as under formula I above, may be prepared by oxidation of compounds of formula IA, wherein n=0, X is SO₂, A, X₁, R₁ and R₄ are defined as under formula I above, under analogous conditions described above.

Alternatively, compounds of formula IC, wherein n=2, X is SO₂, A, X₁, R₁ and R₄ are defined as under formula I above, can be obtained directly by oxidation of compounds of formula IA, wherein n=0, X is SO₂, A, X₁, R₁ and R₄ are defined as under formula I above, under analogous conditions described above.

These reactions can be performed in various organic or aqueous solvents compatible to these conditions, by temperatures from below 0° C. up to the boiling point of the solvent system.

The transformation of compounds of the formula IA into compounds of the formula IB and IC is represented in Scheme 1.

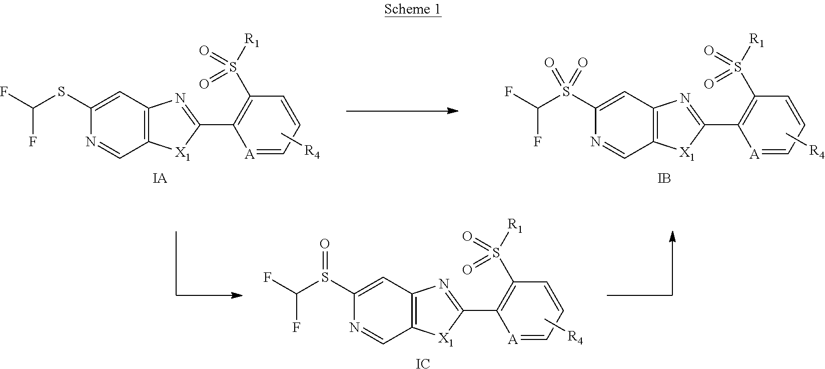

Compounds of formula IA, wherein X is SO₂, A, X₁, R₁ and R₄ are defined as under formula I above, may be prepared as described in Scheme 2.

Compounds of formula IA, wherein n=0, X is SO₂, A, X₁, R₁ and R₄ are defined as under formula I above, may be obtained by treatment of compounds of formula II, wherein X is SO₂, A, X₁, R₁ and R₄ are defined as under formula I above, with a CF2 carbene, generated for example from a difluoro-acetate or a difluoromethyl-phosphonate, wherein X² can be chloro or bromo. Such methods have been described in the literature, e.g. in Org. Lett., 15(19), 5036-5039; 2013 or Tet., 65(27), 5278-5283; 2009.

Alternatively, compounds of formula IA, wherein X is SO₂, A, X₁, R₁ and R₄ are defined as under formula I above, may be obtained by treatment of compounds of formula II, wherein X is SO₂, A, X₁, R₁ and R₄ are defined as under formula I above, with a difluoromethyl-sulfinate, in the presence of an oxidant, as described in J. Fluor. Chem., 193, 113-117; 2017.

Alternatively, compounds of formula IA, wherein X is SO₂, A, X₁, R₁ and R₄ are defined as under formula I above, may be obtained by treatment of compounds of formula II or III, wherein X is SO₂, A, X₁, R₁ and R₄ are defined as under formula I above, with a difluorodiaroylmethane, where Ar may be a substituted phenyl or furane, as described in Org. Lett., 18(3), 592-595; 2016.

Alternatively, compounds of formula IA, wherein X is SO₂, A, X₁, R₁ and R₄ are defined as under formula I above, may be obtained by treatment of compounds of formula III, wherein X is SO₂, A, X₁, R₁ and R₄ are defined as under formula I above, with difluoromethyl(trimethyl)silane, as described in Chem. Comm., 52(54), 8448-8451; 2016 or J. Org. Chem., 81(6), 2506-2512; 2016.

Alternatively, compounds of formula IA, wherein X is SO₂, A, X₁, R₁ and R₄ are defined as under formula I above, may be obtained by treatment of compounds of formula IV, wherein X is SO₂, A, X₁, R₁ and R₄ are defined as under formula I above, and wherein X₃ can be chloro, phenylsulfone or cyano, with difluoromethyl(trimethyl)silane, as described in J. Org. Chem., 81(6), 2506-2512; 2016, or with difluoromethyl(trimethyl)silane, in the presence of copper thiocyanate, as described in Angew. Chem. Int. Ed., 54(19), 5753-5756; 2015 or Chem., Eur. J., 21(41), 14324-14327; 2015.

Alternatively, compounds of formula IA can be prepared from compounds of formula V, wherein X is SO₂, A, X₁, R₁ and R₄ are defined as under formula I above, and wherein X₄ can be bromo, iodo, or triflate, by treatment with sodium thiocyanate, followed by difluoromethyl(trimethyl)silane, in the presence of cesium fluoride and copper thiocyanate, as described in Angew. Chem. Int. Ed., 54(19), 5753-5756; 2015 or Chem., Eur. J., 21(41), 14324-14327; 2015.

Alternatively, compounds of formula IA can be prepared from compounds of formula V, wherein X is SO₂, A, X₁, R₁ and R₄ are defined as under formula I above, and wherein X₄ can be bromo, iodo, or triflate, by treatment with a stabilized silver difluoromethylsulfide reagent, with an heterocyclic carbene, in the presence of a palladium catalyst and a ligand, as described in Chem. Sci., 7(6), 3757-3762; 2016.

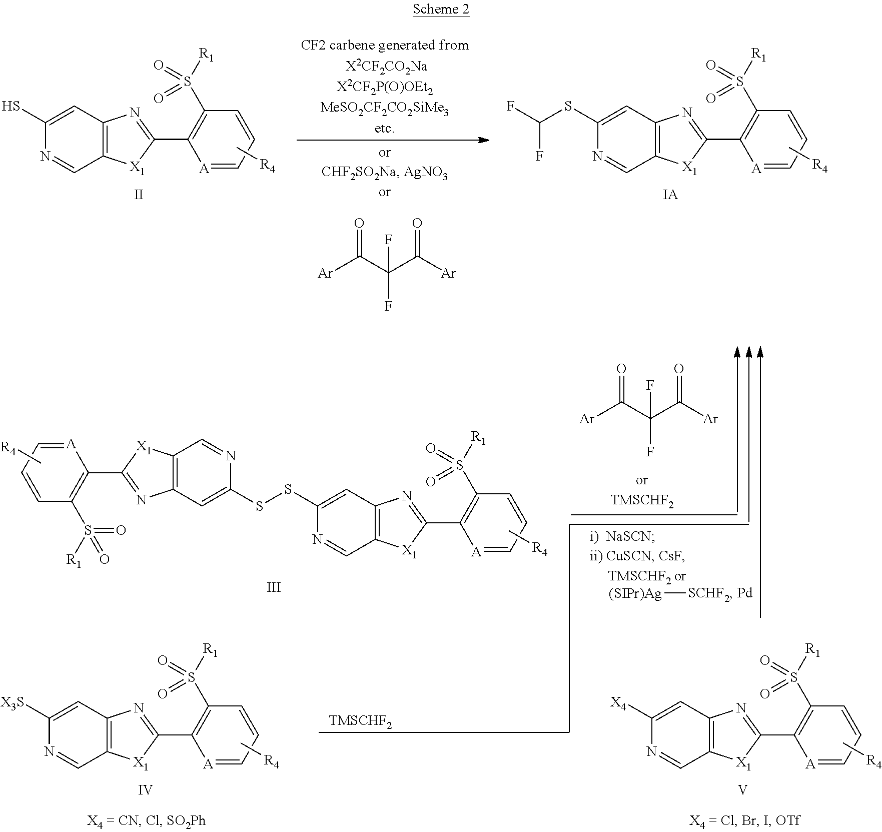

Compounds of formula II, wherein X is SO₂, A, X₁, R₁ and R₄ are defined as under formula I above, may be prepared as described in Scheme 3.

Compounds of formula V, wherein X is SO₂, A, X₁, R₁ and R₄ are defined as under formula I above, and wherein X₄ can be chloro or bromo, may be treated with a sodium or potassium sulfide, in an appropriate solvent like for example N,N-dimethylformamide or ethanol, at temperatures between room temperature and boiling point of the solvent, as described for example in Bioorg. Med. Chem. Lett., 23(13), 3947-3953, 2013 or WO12088190. In a similar way, compounds of formula V, wherein X is SO₂, A, X₁, R₁ and R₄ are defined as under formula I above, and wherein X₄ can be chloro or bromo, may be treated with elemental sulfur in the presence of sodium or potassium hydroxide, as described in Chem. Pharm. Bull., 33(12), 5184-5189, 1985.

Alternatively, compounds of formula V, wherein X is SO₂, A, X₁, R₁ and R₄ are defined as under formula I above, and wherein X₄ can be chloro or bromo, may be treated with sodium or potassium thioacetate in an appropriate solvent for example methanol or water, at temperatures between room temperature and boiling point of the solvent, followed by hydrolysis with sodium or potassium hydroxide, as described for example in WO04018428, or WO12088190.

Alternatively, compounds of formula V, wherein X is SO₂, A, X₁, R₁ and R₄ are defined as under formula I above, and wherein X₄ can be chloro or bromo, may be treated with thiourea in an appropriate solvent for example ethanol, at temperatures between room temperature and boiling point of the solvent, as described for example in Bioorg. Med. Chem. Lett., 23(13), 3947-3953, 2013, J. Med. Chem., 49(2), 727-739; 2006, WO13036464, WO11094209, or WO12088190. Alternatively, compounds of formula V, wherein X is SO₂, A, X₁, R₁ and R₄ are defined as under formula I above, and wherein X₄ can be chloro or bromo, may be treated with sodium thiophosphate in an appropriate solvent for example methanol or water, at temperatures between room temperature and boiling point of the solvent, followed by hydrolysis, as described for example in Tet. Lett., 34(6), 939-942; 1993.

Alternatively, compounds of formula V, wherein X is SO₂, A, X₁, R₁ and R₄ are defined as under formula I above, and wherein X₄ can be bromo or iodo, may be treated with a base like n-butyl lithium in an appropriate solvent for example tetrahydrofuran, at temperatures between −78° C. and room temperature, followed by addition of elemental sulfur, as described for example in WO13036464.

Alternatively, compounds of formula V, wherein X is SO₂, A, X₁, R₁ and R₄ are defined as under formula I above, and wherein X₄ can be bromo, iodo or triflate, may be treated with triisopropylsilanethiol in an appropriate solvent for example toluene, at temperatures between room temperature and boiling point of the solvent, in the presence of a palladium catalyst and a ligand, followed by hydrolysis, as described for example in Adv. Synth. Catal., 347(2-3), 313-319; 2005, or WO13039802.

Alternatively, compounds of formula V, wherein X is SO₂, A, X₁, R₁ and R₄ are defined as under formula I above, and wherein X₄ can be bromo, iodo or triflate, may be treated with p-methoxybenzyl thiol in an appropriate solvent for example dioxane, at temperatures between room temperature and boiling point of the solvent, in the presence of a palladium catalyst and a ligand, and a base, followed by hydrolysis, as described for example in J. Org. Chem., 74(4), 1663-1672; 2009.

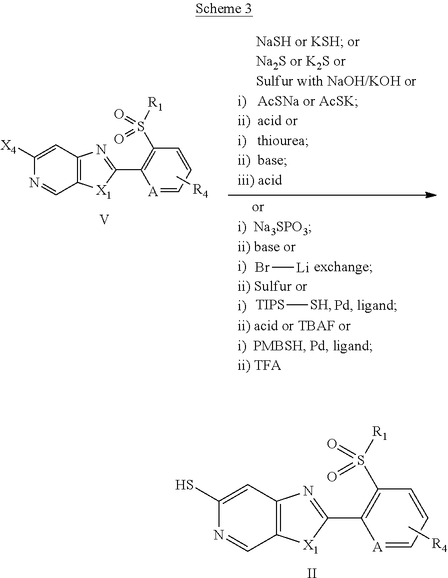

Compounds of formula III, wherein X is SO₂, A, X₁, R₁ and R₄ are defined as under formula I above, may be prepared as described in Scheme 4.

Compounds of formula V, wherein X is SO₂, A, X₁, R₁ and R₄ are defined as under formula I above, and wherein X₄ can be chloro or bromo, may be treated with thiourea in an appropriate solvent for example ethanol, at temperatures between room temperature and boiling point of the solvent, as described for example in Med. Chem. Res., 25(11), 2425-2433; 2016

Numerous examples of synthesis of disulfides from sulfides using different oxidants have been described. Compounds of formula II, wherein X is SO₂, A, X₁, R₁ and R₄ are defined as under formula I above, may be treated with N-chlorosuccinimide, as described for example in Tet. Lett., 56(29), 4305-4307; 2015.

Alternatively, compounds of formula II, wherein X is SO₂, A, X₁, R₁ and R₄ are defined as under formula I above, may be treated with potassium hexacyanoferrate as described for example in Angew. Chem., Int. Ed., 54(1), 280-284; 2015.

Alternatively, compounds of formula II, wherein X is SO₂, A, X₁, R₁ and R₄ are defined as under formula I above, may be treated with sulfuryl chloride as described for example in Tet. Lett., 45(46), 8489-8491; 2004.

Alternatively, compounds of formula II, wherein X is SO₂, A, X₁, R₁ and R₄ are defined as under formula I above, may be treated with diacetoxyiodo-benzene as described for example in Eur. J. Org. Chem., 2014(22), 4795-4804; 2014.

Alternatively, compounds of formula II, wherein X is SO₂, A, X₁, R₁ and R₄ are defined as under formula I above, may be treated with potassium permanganate as described for example in Synth. Comm., 35(4), 571-580; 2005.

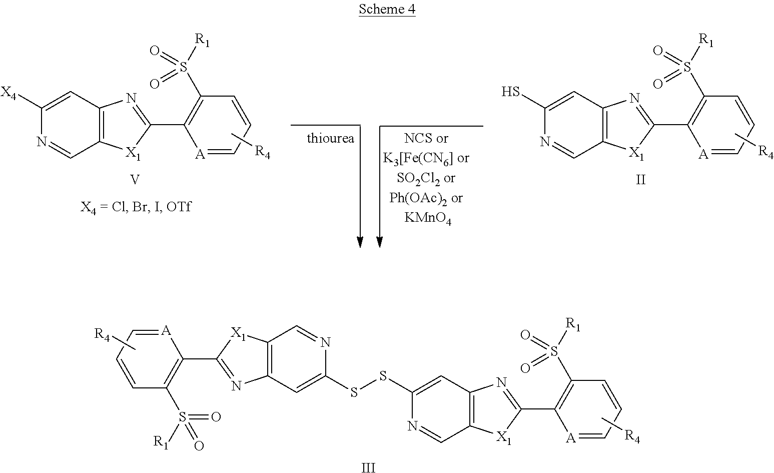

Compounds of formula IV, wherein X is SO₂, A, X₁, R₁ and R₄ are defined as under formula I above, and wherein X₃ is cyano, may be prepared as described in Scheme 5.

Compounds of formula II, wherein X is SO₂, A, X₁, R₁ and R₄ are defined as under formula I above, may be treated with trimethylsilyl cyanide, in the presence of potassium fluoride and a molecular sieve, under oxygen atmosphere, as described in Org. Biomol. Chem., 12(45), 9200-9206; 2014.

Alternatively, compounds of formula II, wherein X is SO₂, A, X₁, R₁ and R₄ are defined as under formula I above, may be treated with p-toluenesulfonyl cyanide, as described in Heterocycles; 45(4), 745-755; 1997.

Alternatively, compounds of formula II, wherein X is SO₂, A, X₁, R₁ and R₄ are defined as under formula I above, may be treated with copper cyanide, in the presence of N,N,N′,N′-tetramethyl-1,2-ethylenediamine, and under air atmosphere, as described in Eur. J. Org. Chem., 2014(35), 7814-7817; 2014. Treatment of compounds of formula III, wherein X is SO₂, A, X₁, R₁ and R₄ are defined as under formula I above, under the same conditions also give compounds of formula IV wherein X₃ is cyano.

Alternatively, compounds of formula III, wherein X is SO₂, A, X₁, R₁ and R₄ are defined as under formula I above, may be treated with iodine in the presence of a base in nitromethane, as described in Tet. Lett., 56(36), 5067-5070; 2015.

Alternatively, compounds of formula III, wherein X is SO₂, A, X₁, R₁ and R₄ are defined as under formula I above, may be treated with azobisisobutyronitrile in the presence of copper iodide and a base, under oxygen atmosphere, as described in Chem. Comm., 50(81), 12139-12141; 2014.

Alternatively, compounds of formula V, wherein X is SO₂, A, X₁, R₁ and R₄ are defined as under formula I above, and wherein X₄ can be bromo, iodo, or triflate, may be treated with sodium thiocyanate as described in Angew. Chem. Int. Ed., 54(19), 5753-5756; 2015.

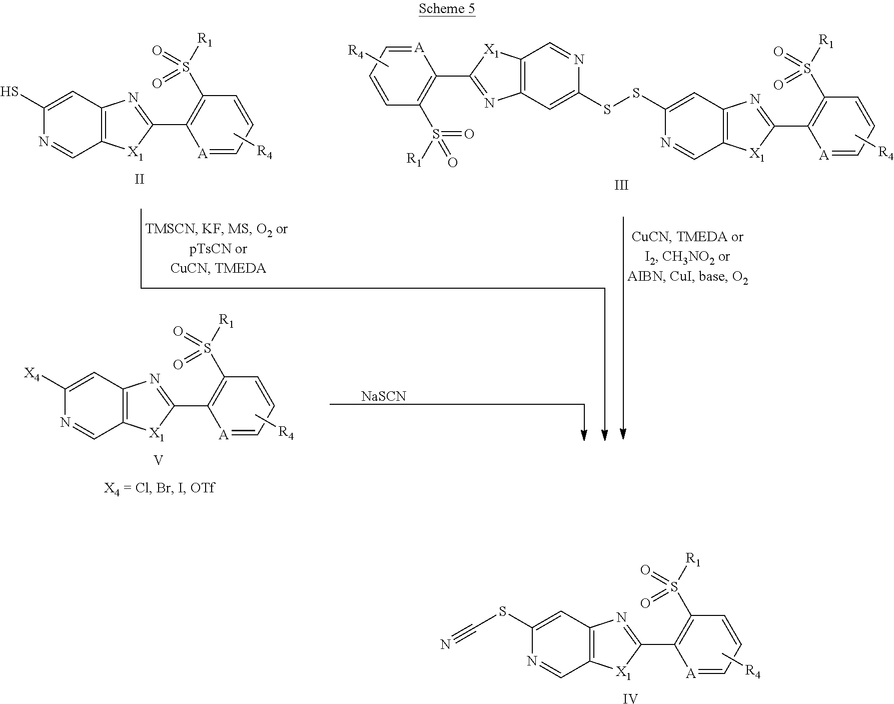

Compounds of formula IV, wherein X is SO₂, A, X₁, R₁ and R₄ are defined as under formula I above, and wherein X₃ is chloro, may be prepared as described in Scheme 6.

Compounds of formula II, wherein X is SO₂, A, X₁, R₁ and R₄ are defined as under formula I above, may be treated with chlorine, as described in J. Org. Chem., 55(9), 2975-2978; 1990.

Alternatively, compounds of formula III, wherein X is SO₂, A, X₁, R₁ and R₄ are defined as under formula I above, may be treated with sulfuryl chloride or thionyl chloride, as described in Tetrahedron Lett., 57(48), 5341-5343; 2016 or J. Agric. Food. Chem., 60, 8286-8293; 2012.

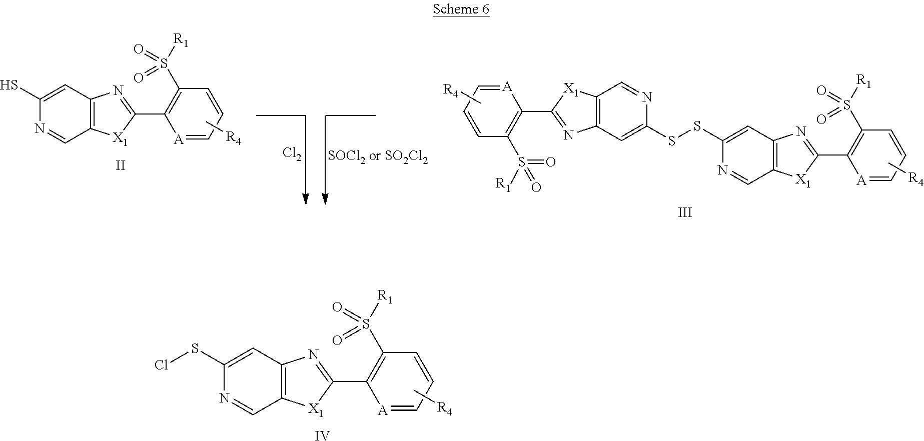

Compounds of formula IV, wherein X is SO₂, A, X₁, R₁ and R₄ are defined as under formula I above, and wherein X₃ is an arylsulfone, where Ar′ may be a substituted phenyl, 2-pyridyl or 1,3-benzoxazol-2-yl, may be prepared as described in Scheme 7.

Compounds of formula II, wherein X is SO₂, A, X₁, R₁ and R₄ are defined as under formula I above, may be treated with an aryl sulfinate in the presence of ferric trichloride, and under air atmosphere, as described in Synlett, 27(12), 1878-1882; 2016. Treatment of compounds of formula III, wherein X is SO₂, A, X₁, R₁ and R₄ are defined as under formula I above, under the same conditions also give compounds of formula IV wherein X₃ is an arylsulfone.

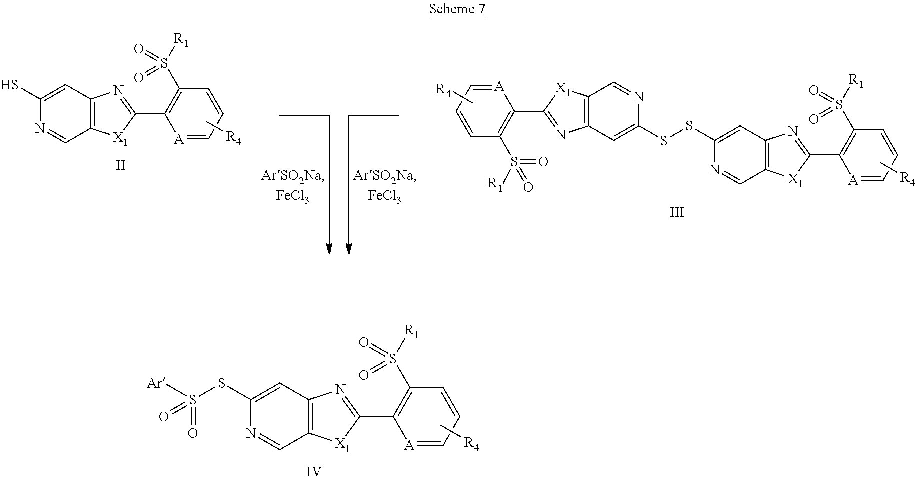

Synthesis of compounds of formula V, wherein X is SO₂, A, X₁, R₁ and R₄ are defined as under formula I above, and wherein X₄ can be chloro, bromo, iodo, or triflate, has been previously described, e.g. in WO 2016/116338.

Alternatively, compounds of formula I may be prepared as described in Scheme 8.

Compounds of formula VI, wherein X, A, R₁ and R₄ are defined as under formula I above, can be activated to compounds of formula VII by methods known to those skilled in the art and described in for example Tetrahedron 2005, 61 (46), 10827-10852. For example compounds of formula VII where X₃ is chlorine are formed by treatment with oxalyl chloride or thionyl chloride in the presence of catalytic quantities of DMF in inert solvents such as methylene chloride or THF at temperatures between 20° C. to 100° C., preferably 25° C. Treatment of VII with compound of formula VIII, wherein X₁ and n are defined as under formula I above, optionally in the presence of a base, e.g. triethylamine or pyridine, leads to compounds of formula IX. Alternatively, compounds of formula VII can be prepared by treatment of compounds of formula VI with dicyclohexyl carbodiimide (DCC) or 1-ethyl-3-(3-dimethylaminopropyl)carbodiimide (EDC) to give the activated species VII, wherein X₃ is VII₀₁ and VII₀₂, respectively, in an inert solvent, e.g. pyridine, or tetrahydrofuran (THF) optionally in the presence of a base, e.g., triethylamine, at temperatures between 50-180° C. The obtained compounds of formula IX can then be converted to compounds of formula IA by dehydration, e.g., by heating under microwave irradiation, in the presence of an acid catalyst, for example methanesulfonic acid, or para-toluenesulfonic acid, in an inert solvent such as N-methyl pyrrolidone at temperatures between 25-180° C., preferably 130-170° C. Such processes have been described previously in WO 2010/125985. Synthesis of the required starting materials of formula VI, wherein X, A, R₁ and R₄ are defined as under formula I above, have been previously described, e.g., in WO 2012/086848, WO 2015/000715, WO 2016/116338, and WO 2016/026848

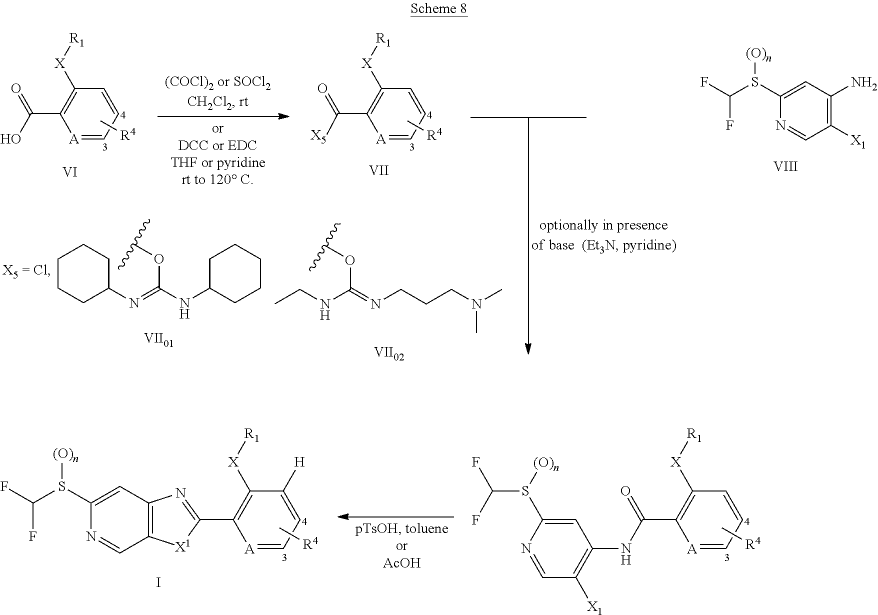

Compounds of formula VIII, wherein X₁ is defined as under formula I above, may be prepared as described in Scheme 9.

Compounds of formula VIII-3, wherein n=2 and X₁ is defined as under formula I above, may be prepared by oxidation of compounds of formula VIII-2, wherein n=1 and X₁ is defined as under formula I above. The reaction can be performed with reagents such as a peracid, for example peracetic acid or m-chloroperbenzoic acid, or a hydroperoxide, as for example, hydrogen peroxide or tert-butylhydroperoxide, or an inorganic oxidant, such as a monoperoxo-disulfate salt or potassium permanganate.

In a similar way, compounds of formula VIII-2, wherein n=1 and X₁ is defined as under formula I above, may be prepared by oxidation of compounds of formula VIII-1, wherein n=0 and X₁ is defined as under formula I above, under analogous conditions described above.

Alternatively, compounds of formula VIII-3, wherein n=2 and X₁ is defined as under formula I above, can be obtained directly by oxidation of compounds of formula VIII-1, wherein n=0 and X₁ is defined as under formula I above, under analogous conditions described above.

These reactions can be performed in various organic or aqueous solvents compatible to these conditions, by temperatures from below 0° C. up to the boiling point of the solvent system.

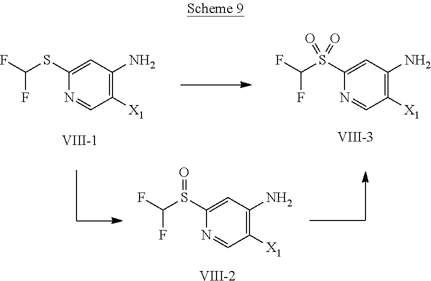

Compounds of formula VIII-1, wherein X₁ is defined as under formula I above, may be prepared as described in Scheme 10.

Compounds of formula VIII-1, wherein X₁ is defined as under formula I above, may be obtained by treatment of compounds of formula X, wherein X₁ is defined as under formula I above, with a CF₂ carbene, generated for example from a difluoro-acetate or a difluoromethyl-phosphonate, wherein X² can be chloro or bromo. Such methods have been described in the literature, e.g. in Org. Lett., 15(19), 5036-5039; 2013 or Tetrahedron, 65(27), 5278-5283; 2009.

Alternatively, compounds of formula VIII-1, wherein X₁ is defined as under formula I above, may be obtained by treatment of compounds of formula XI, wherein n=0 and X₁ is defined as under formula I above, with a difluoromethyl-sulfinate, in the presence of an oxidant, as described in J. Fluor. Chem., 193, 113-117; 2017.

Alternatively, compounds of formula VIII-1, wherein X₁ is defined as under formula I above, may be obtained by treatment of compounds of formula X or XI, wherein X₁ is defined as under formula I above, with a difluorodiaroylmethane, where Ar may be a substituted phenyl or furan, as described in Org. Lett., 18(3), 592-595; 2016.

Alternatively, compounds of formula VIII-1 wherein X₁ is defined as under formula I above, may be obtained by treatment of compounds of formula XI, wherein X₁ is defined as under formula I above, with difluoromethyl(trimethyl)silane, as described in Chem. Comm., 52(54), 8448-8451; 2016 or J. Org. Chem., 81(6), 2506-2512; 2016.

Alternatively, compounds of formula VIII-1, wherein X₁ is defined as under formula I above, may be obtained by treatment of compounds of formula XII, wherein X₁ is defined as under formula I above, and wherein X₃ can be chloro, phenylsulfone or cyano, with difluoromethyl(trimethyl)silane, as described in J. Org. Chem., 81(6), 2506-2512; 2016, or with difluoromethyl(trimethyl)silane, in the presence of copper thiocyanate, as described in Angew. Chem. Int. Ed., 54(19), 5753-5756; 2015 or Chem., Eur. J., 21(41), 14324-14327; 2015.

Alternatively, compounds of formula VIII-1 can be prepared from compounds of formula XIII, wherein X₁ is defined as under formula I above, and wherein X₄ can be bromo, iodo, or triflate, by treatment with sodium thiocyanate, followed by difluoromethyl(trimethyl)silane, in the presence of cesium fluoride and copper thiocyanate, as described in Angew. Chem. Int. Ed., 54(19), 5753-5756; 2015 or Chem., Eur. J., 21(41), 14324-14327; 2015.

Alternatively, compounds of formula VIII-1 can be prepared from compounds of formula XIII, wherein X₁ is defined as under formula I above, and wherein X₄ can be bromo, iodo, or triflate, by treatment with a stabilized silver difluoromethylsulfide reagent, with an heterocyclic carbene, in the presence of a palladium catalyst and a ligand, as described in Chem. Sci., 7(6), 3757-3762; 2016.

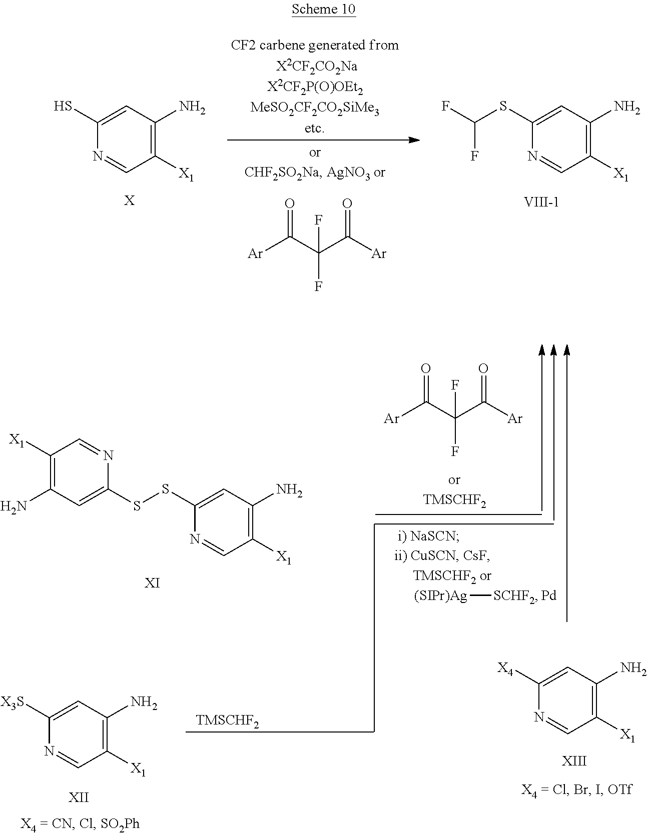

Compounds of formula X, wherein X₁ is defined as under formula I above, may be prepared as described in Scheme 11.

Compounds of formula XIII, wherein X₁ is defined as under formula I above, and wherein X₄ can be chloro or bromo, may be treated with a sodium or potassium sulfide, in an appropriate solvent like for example N,N-dimethylformamide or ethanol, at temperatures between room temperature and boiling point of the solvent, as described for example in Bioorg. Med. Chem. Lett., 23(13), 3947-3953, 2013 or WO12088190. In a similar way, compounds of formula XIII, wherein X₁ is defined as under formula I above, and wherein X₄ can be chloro or bromo, may be treated with elemental sulfur in the presence of sodium or potassium hydroxide, as described in Chem. Pharm. Bull., 33(12), 5184-5189, 1985. Alternatively, compounds of formula XIII, wherein X₁ is defined as under formula I above, and wherein X₄ can be chloro or bromo, may be treated with sodium or potassium thioacetate in an appropriate solvent for example methanol or water, at temperatures between room temperature and boiling point of the solvent, followed by hydrolysis with sodium or potassium hydroxide, as described for example in WO04018428, or WO12088190.

Alternatively, compounds of formula XIII, wherein X₁ is defined as under formula I above, and wherein X₄ can be chloro or bromo, may be treated with thiourea in an appropriate solvent for example ethanol, at temperatures between room temperature and boiling point of the solvent, as described for example in Bioorg.Med. Chem. Lett., 23(13), 3947-3953, 2013, J. Med. Chem., 49(2), 727-739; 2006, WO13036464, WO11094209, or WO12088190.

Alternatively, compounds of formula XIII, wherein X₁ is defined as under formula I above, and wherein X₄ can be chloro or bromo, may be treated with sodium thiophosphate in an appropriate solvent for example methanol or water, at temperatures between room temperature and boiling point of the solvent, followed by hydrolysis, as described for example in Tet. Lett., 34(6), 939-942; 1993.

Alternatively, compounds of formula XIII, wherein X₁ is defined as under formula I above, and wherein X₄ can be bromo, iodo or triflate, may be treated with triisopropylsilanethiol in an appropriate solvent for example toluene, at temperatures between room temperature and boiling point of the solvent, in the presence of a palladium catalyst and a ligand, followed by hydrolysis, as described for example in Adv. Synth. Catal., 347(2-3), 313-319; 2005, or WO13039802.

Alternatively, compounds of formula XIII, wherein X₁ is defined as under formula I above, and wherein X₄ can be bromo, iodo or triflate, may be treated with p-methoxybenzyl thiol in an appropriate solvent for example dioxane, at temperatures between room temperature and boiling point of the solvent, in the presence of a palladium catalyst and a ligand, and a base, followed by hydrolysis, as described for example in J. Org. Chem., 74(4), 1663-1672; 2009.

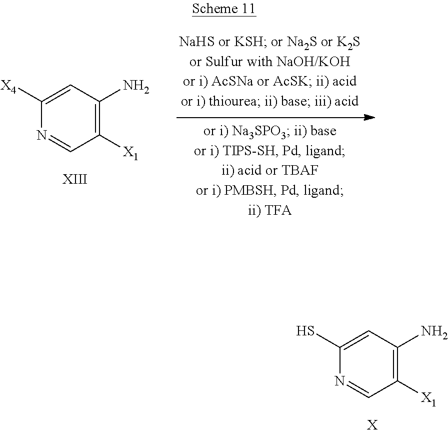

Compounds of formula XI, wherein X₁ is defined as under formula I above, may be prepared as described in Scheme 12.

Compounds of formula XIII, wherein X₁ is defined as under formula I above, and wherein X₄ can be chloro or bromo, may be treated with thiourea in an appropriate solvent for example ethanol, at temperatures between room temperature and boiling point of the solvent, as described for example in Med. Chem. Res., 25(11), 2425-2433; 2016

Numerous examples of synthesis of disulfides from sulfides using different oxidants have been described. Compounds of formula X, wherein X₁ is defined as under formula I above, may be treated with N-chlorosuccinimide, as described for example in Tet. Lett., 56(29), 4305-4307; 2015. Alternatively, formula X, wherein X₁ is defined as under formula I above, may be treated with potassium hexacyanoferrate as described for example in Angew. Chem., Int. Ed., 54(1), 280-284; 2015.

Alternatively, formula X, wherein X₁ is defined as under formula I above, may be treated with diacetoxyiodo-benzene as described for example in Eur. J. Org. Chem., 2014(22), 4795-4804; 2014. Alternatively, compounds of formula X, wherein X₁ is defined as under formula I above, may be treated with sulfuryl chloride as described for example in Tet. Lett., 45(46), 8489-8491; 2004.

Alternatively, compounds of formula X, wherein X₁ is defined as under formula I above, may be treated with potassium permanganate as described for example in Synth. Comm., 35(4), 571-580; 2005.

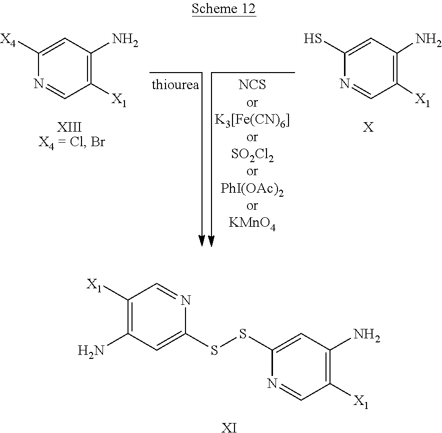

Compounds of formula XII, wherein X₁ is defined as under formula I above, and wherein X₃ is cyano, may be prepared as described in Scheme 13.

Compounds of formula X, wherein X₁ is defined as under formula I above, may be treated with trimethylsilyl cyanide, in the presence of potassium fluoride and a molecular sieve, under oxygen atmosphere, as described in Org. Biomol. Chem., 12(45), 9200-9206; 2014.

Alternatively, compounds of formula X, wherein X₁ is defined as under formula I above, may be treated with p-toluenesulfonyl cyanide, as described in Heterocycles; 45(4), 745-755; 1997.

Alternatively, compounds of formula X, wherein X₁ is defined as under formula I above, may be treated with copper cyanide, in the presence of N,N,N′,N′-tetramethyl-1,2-ethylenediamine, and under air atmosphere, as described in Eur. J. Org. Chem., 2014(35), 7814-7817; 2014. Treatment of compounds of formula XI, wherein X₁ is defined as under formula I above, under the same conditions also give compounds of formula XII wherein X₃ is cyano.

Alternatively, compounds of formula XI, wherein X₁ is defined as under formula I above, may be treated with iodine in the presence of a base in nitromethane, as described in Tet. Lett., 56(36), 5067-5070; 2015.

Alternatively, compounds of formula XI, wherein X₁ is defined as under formula I above, may be treated with azobisisobutyronitrile in the presence of copper iodide and a base, under oxygen atmosphere, as described in Chem. Comm., 50(81), 12139-12141; 2014.

Alternatively, compounds of formula XIII, wherein X₁ is defined as under formula I above, and wherein X₄ can be bromo, iodo, or triflate, may be treated with sodium thiocyanate as described in Angew. Chem. Int. Ed., 54(19), 5753-5756; 2015.

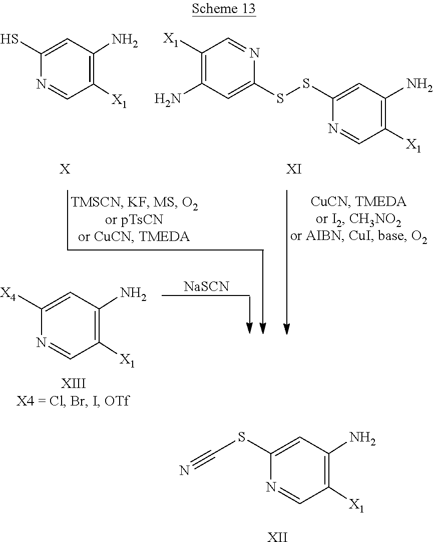

Compounds of formula XII, wherein X₁ is defined as under formula I above, and wherein X₃ is chloro, may be prepared as described in Scheme 14.

Compounds of formula X, wherein X₁ is defined as under formula I above, may be treated with chlorine, as described in J. Org. Chem., 55(9), 2975-2978; 1990.

Alternatively, compounds of formula XI, wherein X₁ is defined as under formula I above, may be treated with sulfuryl chloride or thionyl chloride, as described in Tetrahedron. Lett., 57(48), 5341-5343; 2016 or J. Agric. Food. Chem., 60, 8286-8293; 2012.

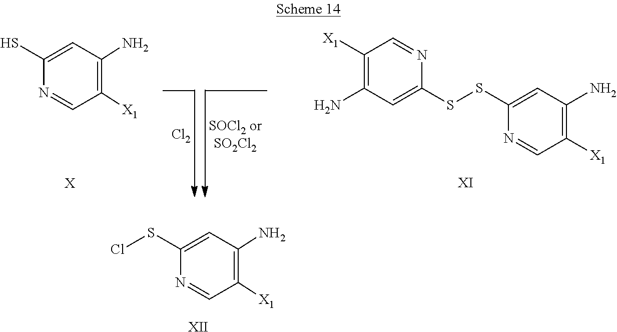

Compounds of formula XII, wherein X₁ is defined as under formula I above, and wherein X₃ is an arylsulfone, where Ar′ may be a substituted phenyl, 2-pyridyl or 1,3-benzoxazol-2-yl, may be prepared as described in Scheme 15.

Compounds of formula X, wherein X₁ is defined as under formula I above, may be treated with an aryl sulfinate in the presence of ferric trichloride, and under air atmosphere, as described in Synlett, 27(12), 1878-1882; 2016. Treatment of compounds of formula XI, wherein X₁ is defined as under formula I above, under the same conditions also give compounds of formula IV wherein X₃ is an arylsulfone.

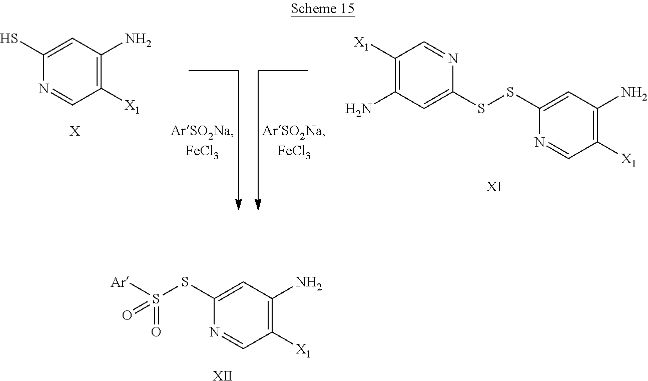

Synthesis of compounds of formula XIII, wherein X₁ is defined as under formula I above, and wherein X₄ can be chloro, bromo, iodo, or triflate, has been previously described, e.g. in WO 2016/116338.

The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reactions are advantageously carried out in a temperature range from approximately −80° C. to approximately +140° C., preferably from approximately −30° C. to approximately +100° C., in many cases in the range between ambient temperature and approximately +80° C.

A compound of formula I can be converted in a manner known per se into another compound of formula I by replacing one or more substituents of the starting compound of formula I in the customary manner by (an)other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula I can be prepared in a manner known per se. Thus, for example, acid addition salts of compounds of formula I are obtained by treatment with a suitable acid or a suitable ion exchanger reagent and salts with bases are obtained by treatment with a suitable base or with a suitable ion exchanger reagent.

Salts of compounds of formula I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula I can be converted in a manner known per se into other salts of compounds of formula I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula I, which have salt-forming properties can be obtained in free form or in the form of salts.

The compounds of formula I and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula I, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

N-oxides can be prepared by reacting a compound of the formula I with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO 00/15615.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula I and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds of Tables X below can be prepared according to the methods described above. The examples which follow are intended to illustrate the invention and show preferred compounds of formula I.

Table X: This table discloses 12 substituent definitions X.001 to X.012 of the formula Ia:

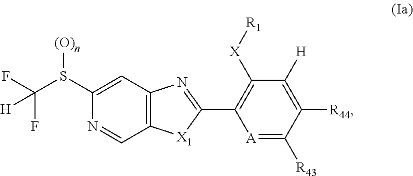

wherein X₁, R₁, A, R₄₃ and R₄₄ are as defined below:

TABLE X

| | X₁ | R₁ | A | R₄₃ | R₄₄ |
|---|---|---|---|---|---|
| X.001 | NMe | Et | N | H | H |
| X.002 | NMe | Et | N | H | CF₃ |
| X.003 | NMe | Et | N | H | Oi-Pr |
| X.004 | NMe | Et | N | H | 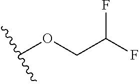 [SMILES: *OCC(F)F] |
| X.005 | NMe | Et | N | H | 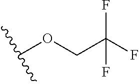 [SMILES: *OCC(F)(F)F] |
| X.006 | NMe | Et | N | H | 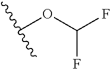 [SMILES: *OC(F)F] |
| X.007 | NMe | Et | N | H | 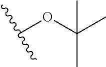 [SMILES: *OC(C)(C)C] |
| X.008 | NMe | Et | N | H | 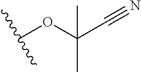 [SMILES: *OC(C)(C)C#N] |
| X.009 | NMe | Et | N | H | 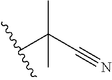 [SMILES: *C(C)(C)C#N] |
| X.010 | NMe | Et | N | H | 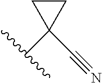 [SMILES: *C1(C#N)CC1] |
| X.011 | NMe | Et | N | 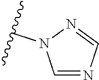 [SMILES: *n1cncn1] | H |
| X.012 | NMe | Et | CH | H | H |

and the N-oxides of the compounds of Table X. Me represents the methyl group, Et is the ethyl group, and i-Pr is the isopropyl group.

Table 1: This table discloses the 12 compounds 1.001 to 1.012 of the formula Ia, wherein n=0, X is S, and X₁, R₁, A, R₄₃ and R₄₄ are as defined in Table X. For example, compound 1.001 has the following structure:

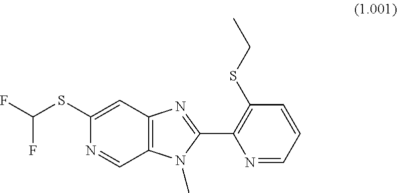 [SMILES: CCSc1cccnc1-c1nc2cc(SC(F)F)ncc2n1C]

Table 2: This table discloses the 12 compounds 2.001 to 2.012 of the formula Ia, wherein n=1, X is S, and X₁, R₁, A, R₄₃ and R₄₄ are as defined in Table X.

Table 3: This table discloses the 12 compounds 3.001 to 3.012 of the formula Ia, wherein n=2, X is S, and X₁, R₁, A, R₄₃ and R₄₄ are as defined in Table X.

Table 4: This table discloses the 12 compounds 4.001 to 4.012 of the formula Ia, wherein n=0, X is SO, and X₁, R₁, A, R₄₃ and R₄₄ are as defined in Table X.

Table 5: This table discloses the 12 compounds 5.001 to 5.012 of the formula Ia, wherein n=1, X is SO, and X₁, R₁, A, R₄₃ and R₄₄ are as defined in Table X.

Table 6: This table discloses the 12 compounds 6.001 to 6.012 of the formula Ia, wherein n=2, X is SO, and X₁, R₁, A, R₄₃ and R₄₄ are as defined in Table X.

Table 7: This table discloses the 12 compounds 7.001 to 4.012 of the formula Ia, wherein n=0, X is SO₂, and X₁, R₁, A, R₄₃ and R₄₄ are as defined in Table X.

Table 8: This table discloses the 12 compounds 8.001 to 8.012 of the formula Ia, wherein n=1, X is SO₂, and X₁, R₁, A, R₄₃ and R₄₄ are as defined in Table X.

Table 9: This table discloses the 12 compounds 9.001 to 9.012 of the formula Ia, wherein n=2, X is 502, and X₁, R₁, A, R₄₃ and R₄₄ are as defined in Table X.

The compounds of formula I according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate.

Examples of the abovementioned animal pests are:

from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,

### Haematopinus spp., Linognathus spp., Pediculus spp., Pemphigus spp. and Phylloxera spp.;

from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example,
Acromyrmex, Arge spp, Atta spp., Cephus spp., Diprion spp., Diprionidae, Gilpinia polytoma, Hoplo-campa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate
from the order Lepidoptera, for example,
Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,

### Damalinea spp. and Trichodectes spp.;

from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,

### Liposcelis spp.;

from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubéreux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. porrum, A. ascalonicum, A. fistulosum), Anthriscus cerefolium, Apium graveolus, Asparagus officinalis, Beta vulgarus, Brassica spp. (B. oleracea, B. pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatoes) and Chilo supressalis (preferably in rice).

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatoes) and Chilo supressalis (preferably in rice).

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by δ-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810). Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit and Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO2006/128870, EP 1724392, WO 2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus | Apple, Pear, Cranberry, |
| | vittaticoillis | Serviceberry, Hawthorn |
| | Chrysobothris | Apple, Apricot, Beech, Boxelder, |
| | femorata | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania | Basswood, Beech, Maple, Oak, |
| | campestris | Sycamore, Willow, Yellow-poplar |
| Cerambycidae | Goes | Beech, Elm, Nuttall, Willow, Black |
| | pulverulentus | oak, Cherrybark oak, Water oak, |
| | | Sycamore |
| | Goes tigrinus | Oak |
| | Neoclytus | Ash, Hickory, Oak, Walnut, Birch, |
| | acuminatus | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes | Fig, Alder, Mulberry, Willow, Netleaf |
| | trilineatus | hackberry |
| | Oberea | Sumac, Apple, Peach, Plum, Pear, |
| | ocellata | Currant, Blackberry |
| | Oberea | Dogwood, Viburnum, Elm, |
| | tripunctata | Sourwood, Blueberry, |
| | | Rhododendron, Azalea, Laurel, |
| | | Poplar, Willow, Mulberry |
| | Oncideres | Hickory, Pecan, Persimmon, Elm, |
| | cingulata | Sourwood, Basswood, Honeylocust, |
| | | Dogwood, Eucalyptus, Oak, |
| | | Hackberry, Maple, Fruit trees |
| | Saperda | Poplar |
| | calcarata | |
| | Strophiona | Chestnut, Oak, Hickory, Walnut, |
| | nitens | Beech, Maple |
| Scolytidae | Corthylus | Maple, Oak, Yellow-poplar, Beech, |
| | columbianus | Boxelder, Sycamore, Birch, |
| | | Basswood, Chestnut, Elm |
| | Dendroctonus | Pine |
| | frontalis | |
| | Dryocoetes | Birch, Sweetgum, Wild cherry, |
| | betulae | Beech, Pear |
| | Monarthrum | Oak, Maple, Birch, Chestnut, |
| | fasciatum | Sweetgum, Blackgum, Poplar, |
| | | Hickory, Mimosa, Apple, Peach, Pine |
| | Phloeotribus | Peach, Cherry, Plum, Black cherry, |
| | liminaris | Elm, Mulberry, Mountain-ash |
| | Pseudo- | Oak, American beech, Black cherry, |
| | pityophthorus | Chickasaw plum, Chestnut, Maple, |
| | pruinosus | Hickory, Hornbeam, Hophornbeam |
| Sesiidae | Paranthrene | Oak, American chestnut |
| | simulans | |
| | Sannina | Persimmon |
| | uroceriformis | |
| | Synanthedon | Peach, Plum, Nectarine, Cherry, |
| | exitiosa | Apricot, Almond, Black cherry |
| | Synanthedon | Peach, Plum, Cherry, Beach, Black |
| | pictipes | Cherry |
| | Synanthedon | Tupelo |
| | rubrofascia | |
| | Synanthedon | Dogwood, Pecan, Hickory, Oak, |
| | scitula | Chestnut, Beech, Birch, Black cherry, |
| | | Elm, Mountain-ash, Viburnum, |
| | | Willow, Apple, Loquat, Ninebark, |
| | | Bayberry |
| | Vitacea | Grape |
| | polistiformis | |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.
Of the order Mallophagida: Trimenopon spp., Menopon spp., Trinoton spp., Bovicola spp., Werneckiella spp., Lepikentron spp., Damalina spp., Trichodectes spp. and Felicola spp.
Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

Of the order Siphonapterida, for example Pulex spp., Ctenocephalides spp., Xenopsylla spp., Ceratophyllus spp.

Of the order Heteropterida, for example Cimex spp., Triatoma spp., Rhodnius spp., Panstrongylus spp.

Of the order Blattarida, for example Blatta orientalis, Periplaneta americana, Blattelagermanica and Supella spp.

Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10th Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance.

Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 l/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

solid carrier: 99.5 to 70%, preferably 97 to 85%

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

| Emulsifiable concentrate | |
|---|---|
| active ingredients | 10% |
| octylphenol polyethylene glycol ether (4-5 mol of | 3% |
| ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol ether (15 mol of | 6% |
| ethylene oxide) | |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form of a 20% | 0.5% |
| solution in water) | |
| monoazo-pigment calcium salt | 5% |
| Silicone oil (in the form of a 75 % emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### PREPARATORY EXAMPLES

“Mp” means melting point in ° C. Free radicals represent methyl groups. ¹H NMR measurements were recorded on a Brucker 400 MHz spectrometer, chemical shifts are given in ppm relevant to a TMS standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻.

### Lcms Methods:

### Method 1: Standard 1

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 I/h, Desolvation Gas Flow: 650 I/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min) 0.85

### Method 2: Standard Long

Spectra were recorded on a Mass Spectrometer from Waters (SQD, SQDII Single quadrupole mass spectrometer) equipped with an electrospray source (Polarity: positive and negative ions), Capillary: 3.00 kV, Cone range: 30V, Extractor: 2.00 V, Source Temperature: 150° C., Desolvation Temperature: 350° C., Cone Gas Flow: 50 I/h, Desolvation Gas Flow: 650 I/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 2.7 min; Flow (ml/min) 0.85

### Method 3: Standard 2 Long

Spectra were recorded on a Mass Spectrometer from Agilent Technologies (6410 Triple Quadrupole mass spectrometer) equipped with an equipped with an electrospray source (Polarity: positive or negative ions, MS2 Scan, Capillary: 4.00 kV, Fragmentor: 100 V, Desolvatation Temperature: 350° C., Gas Flow: 11 L/min, Nebulizer Gas: 45 psi, Mass range: 110 to 1000 Da) and a 1200 Series HPLC from Agilent: pump, heated column compartment and diode-array detector. Column: KINETEX EVO C18, 2.6 μm, 50×4.6 mm, Temp: 40° C., DAD Wavelength range (nm): 210 to 400, Solvent Gradient: A=water+5% Acetonitrile+0.1% HCOOH, B=Acetonitrile+0.1% HCOOH: gradient: 0 min 0% B, 100% A; 0.9-1.8 min 100% B; Flow (ml/min) 1.8.

### Method 4: Standard 2

Spectra were recorded on a Mass Spectrometer from Waters (?) equipped with an electrospray source (Polarity: positive or negative ions, Full Scan, Capillary: 3.00 kV, Cone range: 41 V, Source Temperature: 150° C., Desolvation Temperature: 500° C., Cone Gas Flow: 50 L/Hr, Desolvation Gas Flow: 1000 L/Hr, Mass range: 110 to 800 Da) and a H-Class UPLC from Waters: Binary pump, heated column compartment and diode-array detector. Column: Waters UPLC HSS T3 C18, 1.8 μm, 30×2.1 mm, Temp: 40° C., DAD Wavelength range (nm): 210 to 400, Solvent Gradient: A=water+5% Acetonitrile+0.1% HCOOH, B=Acetonitrile+0.1% HCOOH: gradient: 0 min 10% B; 0-0.2 min 10-50% B; 0.2-0.7 min 50-100% B; Flow (ml/min) 0.8.

### Example P1: Preparation of 6-(difluoromethylsulfonyl)-2-[3-ethylsulfonyl-5-(trifluoromethyl)-2-pyridyl]-3-methyl-imidazo[4,5-c]pyridine (Compound P1)

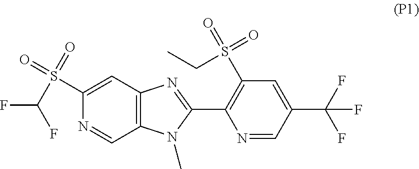 [SMILES: CCS(=O)(=O)c1cc(C(F)(F)F)cnc1-c1nc2cc(S(=O)(=O)C(F)F)ncc2n1C]

### Step 1: Preparation of 2-[3-ethylsulfonyl-5-(trifluoromethyl)-2-pyridyl]-6-[(4-methoxyphenyl)methylsulfanyl]-3-methyl-imidazo[4,5-c]pyridine (Compound I1)

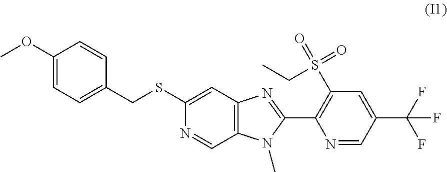 [SMILES: CCS(=O)(=O)c1cc(C(F)(F)F)cnc1-c1nc2cc(SCc3ccc(OC)cc3)ncc2n1C]

6-bromo-2-[3-ethylsulfonyl-5-(trifluoromethyl)-2-pyridyl]-3-methyl-imidazo[4,5-b]pyridine (500 mg, 1.11 mmol, 1.00 equiv., prepared as described in WO 2013018928), (4-methoxyphenyl)methanethiol (170 μL, 1.22 mmol, 1.10 equiv.) and diisopropyl-ethylamine (380 μL, 2.22 mmol, 2.00 equiv.) were introduced into a microwave vial and dissolved in dioxane (11.1 mL). After flushing the reaction mixture with argon for 5 min, 3-chloroprop-1-ene; (5-diphenylphosphanyl-9,9-dimethyl-xanthen-4-yl)-diphenyl-phosphane; palladium (42 mg, 0.055 mmol, 0.05 equiv.) was added, the vial was closed, and the yellow mixture was heated at 90° C. After stirring overnight, the reaction mixture was cooled down to room temperature, quenched with water, and the aqueous phase was extracted twice with ethyl acetate. The combined organic phases were washed with water, dried over sodium sulfate, filtered and concentrated. The crude material was purified by flash chromatography over silica gel, eluting with ethyl acetate in cyclohexane, providing the expected product as a yellow solid. LCMS (method 1): 524 (M+2H)⁺; retention time: 1.08 min.

### Step 2: Preparation of 2-[3-ethylsulfonyl-5-(trifluoromethyl)-2-pyridyl]-3-methyl-imidazo[4,5-c]pyridine-6-thiol (compound 12)

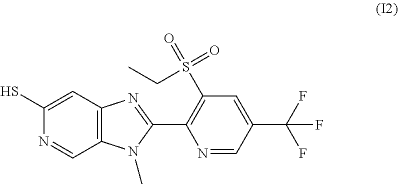 [SMILES: CCS(=O)(=O)c1cc(C(F)(F)F)cnc1-c1nc2cc(S)ncc2n1C]

A yellow solution of 2-[3-ethylsulfonyl-5-(trifluoromethyl)-2-pyridyl]-6-[(4-methoxyphenyl)methylsulfanyl]-3-methyl-imidazo[4,5-c]pyridine (498 mg, 0.95 mmol, compound I1) in trifluoroacetic acid (4.8 mL), was heated at 75° C. and stirred overnight. After cooling down to room temperature, the reaction mixture was directly evaporated. The crude material (containing some disulfide) was directly engaged in the next step. LCMS (method 1): expected product 403 (M+H)⁺; retention time: 0.71 min; disulfide 803 (M+H)⁺; retention time: 1.12 min.

### Step 3: Preparation of 6-(difluoromethylsulfanyl)-2-[3-ethylsulfonyl-5-(trifluoromethyl)-2-pyridyl]-3-methyl-imidazo[4,5-c]pyridine (Compound P3)

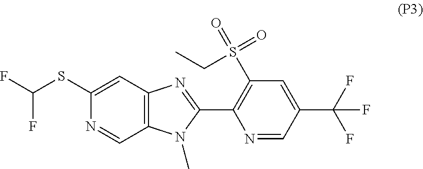 [SMILES: CCS(=O)(=O)c1cc(C(F)(F)F)cnc1-c1nc2cc(SC(F)F)ncc2n1C]

Potassium carbonate (801 mg, 5.71 mmol, 6.00 equiv.) was added to a brown solution of crude 2-[3-ethylsulfonyl-5-(trifluoromethyl)-2-pyridyl]-3-methyl-imidazo[4,5-c]pyridine-6-thiol (383 mg, 0.95 mmol, obtained above) in N,N-dimethylformamide (4.8 mL). Sodium chlorodifluoroacetate (292 mg, 1.90 mmol, 2.00 equiv.) was added portionwise, and the reaction mixture was heated to 95° C. and stirred overnight. After cooling down to room temperature, the reaction was quenched with water, and the aqueous phase was extracted twice with ethyl acetate. The combined organic phases were washed four times with water, dried over sodium sulfate, filtered and evaporated. Purification of the crude material by flash chromatography over silica gel, eluting with ethyl acetate in cyclohexane, afforded the desired product as a yellow solid. LCMS (method 1): 453 (M+H)⁺; retention time: 1.01 min.

### Step 4: Preparation of 6-(difluoromethylsulfonyl)-2-[3-ethylsulfonyl-5-(trifluoromethyl)-2-pyridyl]-3-methyl-imidazo[4,5-c]pyridine (Compound P1)

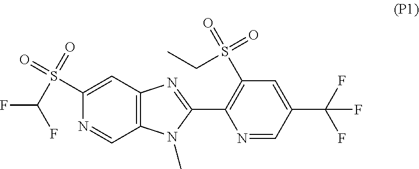 [SMILES: CCS(=O)(=O)c1cc(C(F)(F)F)cnc1-c1nc2cc(S(=O)(=O)C(F)F)ncc2n1C]

Sodium periodate (452 mg, 2.11 mmol, 4.60 equiv.) followed by trichlororuthenium hydrate (6.0 mg, 0.011 mmol, 0.025 equiv.) were added to a suspension of 6-(difluoromethylsulfanyl)-2-[3-ethylsulfonyl-5-(trifluoromethyl)-2-pyridyl]-3-methyl-imidazo[4,5-c]pyridine (208 mg, 0.460 mmol, 1.00 equiv., compound P3) in a mixture of acetonitrile (690 μL), carbon tetrachloride (690 μL) and water (1.38 mL). The reaction mixture was stirred at room temperature for 2 hours, then diluted with water and extracted several times with dichloromethane. The combined organic phases were dried over sodium sulfate, filtered and concentrated. The crude material was purified by flash chromatography over silica gel, eluting with ethyl acetate in cyclohexane, to afford the desired product as a white foam. LCMS (method 1): 485 (M+H)⁺; retention time: 0.95 min.

### Example P2: Preparation of 6-(difluoromethylsulfinyl)-2-[3-ethylsulfonyl-5-(trifluoromethyl)-2-pyridyl]-3-methyl-imidazo[4,5-c]pyridine (Compound P2)

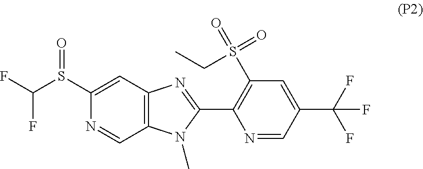 [SMILES: CCS(=O)(=O)c1cc(C(F)(F)F)cnc1-c1nc2cc(S(=O)C(F)F)ncc2n1C]

3-chloroperbenzoic acid (38 mg, 0.221 mmol, 1.00 equiv.) was added portionwise to a 0° C. cooled solution of 6-(difluoromethylsulfanyl)-2-[3-ethylsulfonyl-5-(trifluoromethyl)-2-pyridyl]-3-methyl-imidazo[4,5-c]pyridine (compound P3, prepared as described above for example P1, 100 mg, 0.221 mmol, 1.00 equiv.) in dichloromethane (1.8 mL). After stirring for 1 hour at room temperature, the reaction mixture was poured over a sodium thioslufate solution. The phases were separated, and the aqueous phase was extracted twice with ethyl acetate. The combined organic phase were washed with water, dried over sodium sulfate, filtered and concentrated after a peroxide test confirming the disappearance of any peroxides. Purification by flash chromatography over silica gel, eluting with ethyl acetate in dichloromethane, afforded the desired product as a white foam. LCMS (method 1): 469 (M+H)⁺; retention time: 0.89 min.

### Example P4: Preparation of 6-(difluoromethylsulfinyl)-2-(3-ethylsulfonyl-2-pyridyl)-3-methyl-imidazo[4,5-c]pyridine (Compound P4)

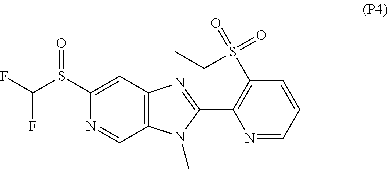 [SMILES: CCS(=O)(=O)c1cccnc1-c1nc2cc(S(=O)C(F)F)ncc2n1C]

### Step 1: Preparation of N-[2-bromo-5-(methylamino)-4-pyridyl]-3-ethylsulfanyl-pyridine-2-carboxamide and N-(4-amino-6-bromo-3-pyridyl)-3-ethylsulfanyl-N-methyl-pyridine-2-carboxamide (Compounds 13)

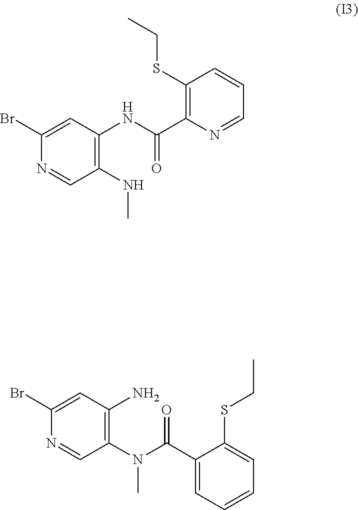 [SMILES: CCSc1cccnc1C(=O)Nc1cc(Br)ncc1NC ; CCSc1ccccc1C(=O)N(C)c1cnc(Br)cc1N]

Oxalyl chloride (4.61 g, 3.07 mL, 36.3 mmol, 1.21 equiv.) was added dropwise to a solution of 3-ethylsulfanylpyridine-2-carboxylic acid (6.05 g, 33 mmol, 1.1 equiv., prepared as described in WO 2013018928) in DCM (165 mL) containing a catalytic amount of N,N-dimethylformamide (128 μL, 1.65 mmol, 0.055 equiv.). The reaction mixture was stirred for 1.5 hours. The dark solution was concentrated under vacuum and used directly.

A suspension of 3-ethylsulfanylpyridine-2-carbonyl chloride (6.65 g, 33 mmol, 1.1 equiv., obtained above) in tetrahydrofuran (102 mL) was added slowly to a 0° C. cooled solution of 6-bromo-N3-methyl-pyridine-3,4-diamine (6.06 g, 30 mmol, 1.0 equiv., prepared as described in WO 2016/107831) and Hunig's base (5.82 g, 7.70 mL, 45 mmol, 1.5 equiv.) in tetrahydrofuran (120 mL). The reaction mixture was stirred for 30 min after complete addition. The mixture was then poured over water, and the aqueous phase was extracted with ethyl acetate (brine was added to avoid an emulsion). The combined organic phases were dried over sodium sulfate, filtered and concentrated. The crude material was used in the next step directly without purification. LCMS (method 1): 367, 369 (M+H)⁺; retention time: 0.74 min.

### Step 2: Preparation of 6-bromo-2-(3-ethylsulfanyl-2-pyridyl)-3-methyl-imidazo[4,5-c]pyridine

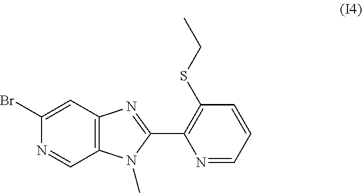 [SMILES: CCSc1cccnc1-c1nc2cc(Br)ncc2n1C]

A solution of N-[2-bromo-5-(methylamino)-4-pyridyl]-3-ethylsulfanyl-pyridine-2-carboxamide and N-(4-amino-6-bromo-3-pyridyl)-3-ethylsulfanyl-N-methyl-pyridine-2-carboxamide (crude, 3.90 g, 11.0 mmol) in acetic acid (20 mL) was stirred at 120° C. overnight. The mixture was cooled down to room temperature, poured over water, and the aqueous phase was extracted with ethyl acetate. The combined organic phases were dried over sodium sulfate, filtered and concentrated. The residue was purified by flash chromatography on silica gel eluting with ethyl acetate in cyclohexane. The selected fractions were combined and evaporated to afford the title compound as a beige solid. LCMS (method 1): 349, 351 (M+H)⁺; retention time: 0.86 min.

### Step 3: Preparation of 6-bromo-2-(3-ethylsulfonyl-2-pyridyl)-3-methyl-imidazo[4,5-c]pyridine (Compound 15)

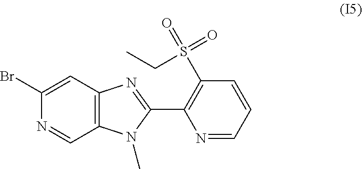 [SMILES: CCS(=O)(=O)c1cccnc1-c1nc2cc(Br)ncc2n1C]

3-chloroperbenzoic acid (7.77 g, 31.5 mmol, 2.20 equiv.) was added portionwise to a solution of 6-bromo-2-(3-ethylsulfanyl-2-pyridyl)-3-methyl-imidazo[4,5-c]pyridine (5.00 g, 14.3 mmol, 1.00 equiv.) in dichloromethane (100 mL) under argon. The temperature was maintained below 30° C. with ice cooling. After complete addition, the reaction mixture was stirred at room temperature overnight. The reaction mixture was slowly poured over a saturated sodium hydrogenocarbonate solution, the aqueous phase was extracted three times with dichloromethane. The combined organic phases were washed with a saturated sodium hydrogenocarbonate solution, and then a 10% w/v sodium metabisulfite aqueous solution, dried over sodium sulfate, filtered and concentrated. Purification of the crude material by flash chromatography over silica gel, eluting with ethyl acetate in cyclohexane afforded the desired product as a yellow solid. LCMS (method 1): 381 (383) (M)⁺; retention time: 0.76 min.

### Step 4: Preparation of 2-(3-ethylsulfonyl-2-pyridyl)-3-methyl-imidazo[4,5-c]pyridine-6-thiol (Compound 16)

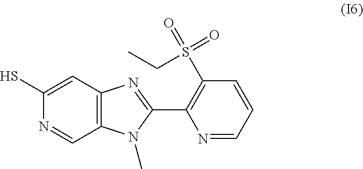 [SMILES: CCS(=O)(=O)c1cccnc1-c1nc2cc(S)ncc2n1C]

Palladium acetate (11 mg, 0.067 mmol, 0.05 equiv.), triphenylphosphine (76 mg, 0.289 mmol, 0.22 equiv.), cesium carbonate (329 mg, 1.71 mmol, 1.30 equiv.) 6-bromo-2-(3-ethylsulfonyl-2-pyridyl)-3-methyl-imidazo[4,5-c]pyridine (500 mg, 1.31 mmol, 1.00 equiv.) were introduced in a 30 mL supelco vial. The vial was evacuated and backfilled with argon three times. Toluene (13 mL) and triisopropylsilanethiol (325 mg, 366 μL, 1.71 mmol, 1.30 equiv.) were added, and the vial was heated to 110° C. and stirred for 1.5 hours. After cooling down to room temperature, the reaction mixture was directly evaporated over silica gel and purified by column chromatography over silica gel, eluting with methanol in dichloromethane. The selected fractions were combined and evaporated to yield the desired product as a yellow solid. The product was kept in the freezer overnight and enganged in the next step. LCMS (method 1): 335 (M+H)⁺; retention time: 0.29 min.

### Step 5: Preparation of 6-(difluoromethylsulfanyl)-2-(3-ethylsulfonyl-2-pyridyl)-3-methyl-imidazo[4,5-c]pyridine (Compound P6)

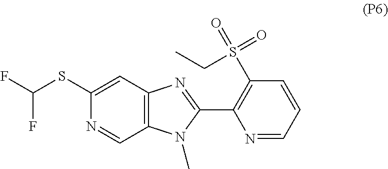 [SMILES: CCS(=O)(=O)c1cccnc1-c1nc2cc(SC(F)F)ncc2n1C]

Potassium carbonate (198 mg, 1.96 mmol, 1.50 equiv.), sodium 2-chloro-2,2-difluoro-acetate (398 mg, 2.61 mmol, 2.00 equiv.) and 2-(3-ethylsulfonyl-2-pyridyl)-3-methyl-imidazo[4,5-c]pyridine-6-thiol (437 mg, 1.31 mmol, 1.00 equiv.) were introduced in a 30 mL supelco vial. The vial was evacuated and backfilled with argon three times, before adding N,N-dimethylformamide (6.5 mL), and then heated in a preheated alublock at 95° C. After cooling down to room temperature, the reaction mixture was diluted with ethyl acetate and the organic phase was washed with water. The aqueous phase was extracted twice with ethyl acetate. The combined organic phases were washed with water, brine, and dried over sodium sulfate, filtered and concentrated. The crude material was purified by flash chromatography over silica gel, eluting with methanol in dichloromethane, to afford the desired product as a yellow solid. LCMS (method 1): 385 (M+H)⁺; retention time: 0.84 min.

### Step 6: Preparation of 6-(difluoromethylsulfinyl)-2-(3-ethylsulfonyl-2-pyridyl)-3-methyl-imidazo[4,5-c]pyridine (Compound P4)

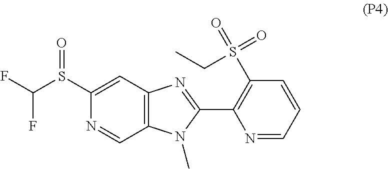 [SMILES: CCS(=O)(=O)c1cccnc1-c1nc2cc(S(=O)C(F)F)ncc2n1C]

3-chloroperbenzoic acid (68.4 mg, 0.278 mmol, 1.10 equiv.) was added portionwise to a 0° C. cooled solution of 6-(difluoromethylsulfanyl)-2-(3-ethylsulfonyl-2-pyridyl)-3-methyl-imidazo[4,5-c]pyridine (97 mg, 0.252 mmol, 1.00 equiv.) in dichloromethane (1.9 mL). After stirring for 3 hours at room temperature, the reaction mixture was poured over a saturated sodium hydrogenocarbonate solution.

The phases were separated, the organic phase washed again with a saturated sodium hydrogenocarbonate solution. The combined aqueous phases were extracted with dichloromethane, the combined organic phases were washed with a 40% sodium bisulfite solution, dried over sodium sulfate, filtered and concentrated. Two purifications by flash chromatography over silica gel, eluting with methanol in dichloromethane, afforded the desired product as a white solid. LCMS (method 1): 401 (M+H)⁺; retention time: 0.68 min.

### Example P5: Preparation of 6-(difluoromethylsulfonyl)-2-(3-ethylsulfonyl-2-pyridyl)-3-methyl-imidazo[4,5-c]pyridine (Compound P5)

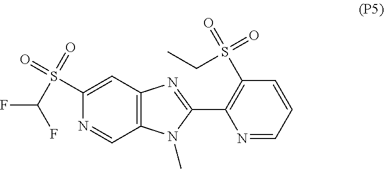 [SMILES: CCS(=O)(=O)c1cccnc1-c1nc2cc(S(=O)(=O)C(F)F)ncc2n1C]

Sodium periodate (256 mg, 1.20 mmol, 4.60 equiv.) followed by trichlororuthenium hydrate (15 mg, 0.0065 mmol, 0.0250 equiv.) were added to a suspension of 6-(difluoromethylsulfanyl)-2-(3-ethylsulfonyl-2-pyridyl)-3-methyl-imidazo[4,5-c]pyridine (compound P6, prepared as described above in step 5 for example P4, 100 mg, 0.260 mmol, 1.00 equiv.) in a mixture of acetonitrile (336 μL), carbon tetrachloride (336 μL) and water (838 μL). The reaction mixture was stirred at room temperature for 2.5 hours, then diluted with water and extracted several times with dichloromethane.

The combined organic phases were dried over sodium sulfate, filtered and concentrated. The crude material was purified by flash chromatography over silica gel, eluting with ethyl acetate in cyclohexane, to afford the desired product as a white solid. LCMS (method 1): 417 (M+H)⁺; retention time: 0.78 min.

### Example P7: Preparation of 6-(difluoromethylsulfonyl)-2-[3-ethylsulfonyl-6-(1,2,4-triazol-1-yl)-2-pyridyl]-3-methyl-imidazo[4,5-c]pyridine (Compound P7)

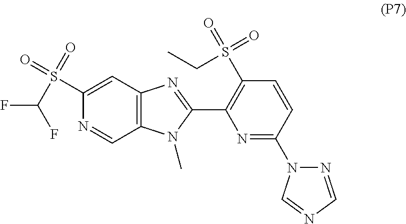 [SMILES: CCS(=O)(=O)c1ccc(-n2cncn2)nc1-c1nc2cc(S(=O)(=O)C(F)F)ncc2n1C]

### Step 1: Preparation of methyl 3-ethylsulfonyl-6-(1,2,4-triazol-1-yl)pyridine-2-carboxylate (Compound 17)

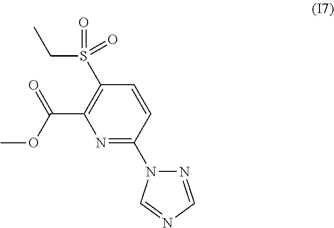 [SMILES: CCS(=O)(=O)c1ccc(-n2cncn2)nc1C(=O)OC]

A solution of methyl 6-chloro-3-ethylsulfonyl-pyridine-2-carboxylate (prepared as described in WO 2016/116338, 5.0 g, 19 mmol, 1.0 equiv)) in N-methyl-pyrrolidone (5.0 mL), was added dropwise to a grey suspension of sodium hydride (2.2 g, 57 mmol, 3.0 equiv.) and 4H-1,2,4-triazole (4.7 g, 68 mmol, 3.6 equiv.) in N-methyl-pyrrolidone (5.0 mL). After stirring for 1 hour at room temperature, the reaction mixture was carefully quenched with methanol, followed by addition of water. The white precipitate was filtered and dried under vacuum. Trituration in diethyl ether afforded the desired compound as a yellow solid. LCMS (method 1): 297 (M+H)⁺; retention time: 0.67 min.

### Step 2: Preparation of 3-ethylsulfonyl-6-(1,2,4-triazol-1-yl)pyridine-2-carboxylic acid (Compound 18)

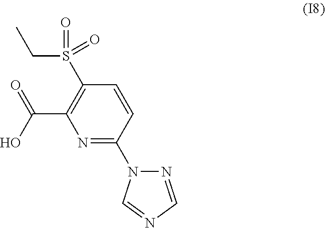 [SMILES: CCS(=O)(=O)c1ccc(-n2cncn2)nc1C(=O)O]

Lithium hydroxide (500 mg, 20.9 mmol, 1.20 equiv.) was added to a solution of methyl 3-ethylsulfonyl-6-(1,2,4-triazol-1-yl)pyridine-2-carboxylate (compound 17 prepared above, 5.16 g, 17.4 mmol, 1.00 equiv.) in tetrahydrofuran (44 mL) and water (9 mL). After stirring for 2 hours at room temperature, the reaction mixture was diluted with water, and the pH was acidified to 2 by adding a 2M hydrochloric acid aqueous solution. The aqueous phase was extracted with ethyl acetate, the combined organic phases were washed with water, dried over sodium sulfate, filtered and concentrated. The crude material was engaged in the next step directly. LCMS (method 1): 283 (M+H)⁺; retention time: 0.26 min.

### Step 3: Preparation of N-[2-bromo-5-(methylamino)-4-pyridyl]-3-ethylsulfonyl-6-(1,2,4-triazol-1-yl)pyridine-2-carboxamide and N-(4-amino-6-bromo-3-pyridyl)-3-ethylsulfonyl-N-methyl-6-(1,2,4-triazol-1-yl)pyridine-2-carboxamide (compounds 19)

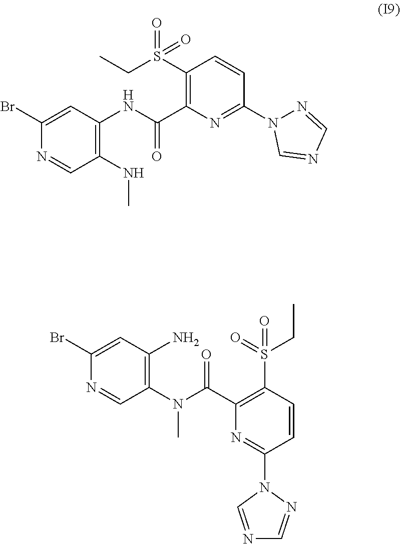 [SMILES: CCS(=O)(=O)c1ccc(-n2cncn2)nc1C(=O)Nc1cc(Br)ncc1NC ; CCS(=O)(=O)c1ccc(-n2cncn2)nc1C(=O)N(C)c1cnc(Br)cc1N]

Oxalyl chloride (834 μL, 9.56 mmol, 1.50 equiv.) was added dropwise to a solution of 3-ethylsulfonyl-6-(1,2,4-triazol-1-yl)pyridine-2-carboxylic acid (compound 18 prepared above, 1.80 g, 6.38 mmol, 1.00 equiv.) in DCM (41 mL) containing a catalytic amount of N,N-dimethylformamide. The reaction mixture was stirred for 5 hours. The dark solution was concentrated under vacuum and used directly.

A suspension of 3-ethylsulfonyl-6-(1,2,4-triazol-1-yl)pyridine-2-carbonyl chloride (1.80 g, 5.99 mmol 1.10 equiv., obtained above) in tetrahydrofuran (35 mL) was added slowly to a 0° C. cooled solution of 6-bromo-N3-methyl-pyridine-3,4-diamine (1.10 g, 5.44 mmol, 1.00 equiv., prepared as described in WO 2016107831) and Hunig's base (2.38 mL, 13.6 mmol, 2.5 equiv.) in tetrahydrofuran (35 mL). The reaction mixture was stirred for 30 min at 0° C. and overnight at room temperature. The mixture was then quenched with a saturated ammonium chloride aqueous solution, and the aqueous phase was extracted with ethyl acetate. The combined organic phases were washed with water, brine, dried over sodium sulfate, filtered and concentrated. The crude material was used in the next step directly without purification. LCMS (method 1): 466, 468 (M+H)⁺; retention time: 0.71 min.

### Step 4: Preparation of 6-bromo-2-[3-ethylsulfonyl-6-(1,2,4-triazol-1-yl)-2-pyridyl]-3-methyl-imidazo[4,5-c]pyridine (Compound 110)

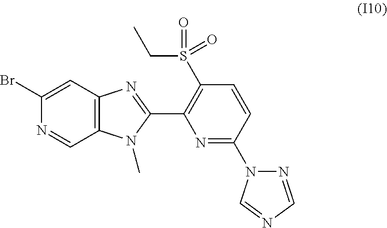 [SMILES: CCS(=O)(=O)c1ccc(-n2cncn2)nc1-c1nc2cc(Br)ncc2n1C]

A solution of crude N-[2-bromo-5-(methylamino)-4-pyridyl]-3-ethylsulfonyl-6-(1,2,4-triazol-1-yl)pyridine-2-carboxamide and N-(4-amino-6-bromo-3-pyridyl)-3-ethylsulfonyl-N-methyl-6-(1,2,4-triazol-1-yl)pyridine-2-carboxamide (compounds 19 prepared above, 2.37 g, 5.08 mmol) in acetic acid (15 mL) was heated at 100° C. and stirred for 5 hours. After cooling down to room temperature, the reaction mixture was concentrated, evaporated again with toluene. Two purifications by flash chromatography over silica gel, eluting with methanol in dichloromethane, afforded the desired compound. LCMS (method 1): 448, 450 (M+H)⁺; retention time: 0.82 min.

### Step 5: Preparation of 2-[3-ethylsulfonyl-6-(1,2,4-triazol-1-yl)-2-pyridyl]-6-[(4-methoxyphenyl)methylsulfanyl]-3-methyl-imidazo[4,5-c]pyridine (Compound 111)

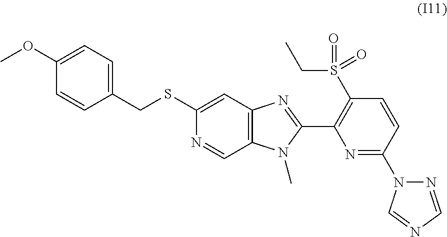 [SMILES: CCS(=O)(=O)c1ccc(-n2cncn2)nc1-c1nc2cc(SCc3ccc(OC)cc3)ncc2n1C]

After degassing a mixture of 6-bromo-2-[3-ethylsulfonyl-6-(1,2,4-triazol-1-yl)-2-pyridyl]-3-methyl-imidazo[4,5-c]pyridine (compound 110 prepared above, 1.20 g, 2.68 mmol, 1.00 equiv.), 4-methoxybenzyl mercaptan (408 μL, 2.94 mmol, 1.10 equiv.) and Hunig's base (932 μL, 5.35 mmol, 2.00 equiv.) in 1,4-dioxane (27 mL) with argon for 5 min, 3-chloroprop-1-ene-(5-diphenylphosphanyl-9,9-dimethyl-xanthen-4-yl)-diphenyl-phosphane; palladium (102 mg, 0.134 mmol, 0.05 equiv.) was added portionwise. The reaction mixture was heated at 90° C. and stirred for 8 hours. After cooling down to room temperature, the mixture was quenched by addition of water, and the aqueous phase was extracted twice with ethyl acetate. The combined organic phases were washed four times with water, dried over sodium sulfate, filtered and concentrated. The crude material was used in the next step directly. LCMS (method 1): 522 (M+H)⁺; retention time: 0.96 min.

### Step 6: Preparation of 2-[3-ethylsulfonyl-6-(1,2,4-triazol-1-yl)-2-pyridyl]-3-methyl-imidazo[4,5-c]pyridine-6-thiol (Compound 112)

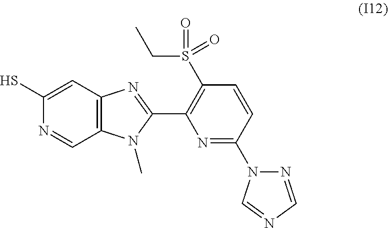 [SMILES: CCS(=O)(=O)c1ccc(-n2cncn2)nc1-c1nc2cc(S)ncc2n1C]

A mixture of 2-[3-ethylsulfonyl-6-(1,2,4-triazol-1-yl)-2-pyridyl]-6-[(4-methoxyphenyl)methylsulfanyl]-3-methyl-imidazo[4,5-c]pyridine (compound 111 prepared above, 1.10 g, 2.11 mmol) in trifluoroacetic acid (11 mL) was heated at 75° C. and stirred overnight. After cooling down to room temperature, the reaction mixture was concentrated under vacuum. The crude material was used in the next step directly. LCMS (method 1): 402 (M+H)⁺; retention time: 0.55 min.

### Step 7: Preparation of 6-(difluoromethylsulfanyl)-2-[3-ethylsulfonyl-6-(1,2,4-triazol-1-yl)-2-pyridyl]-3-methyl-imidazo[4,5-c]pyridine (Compound P9)

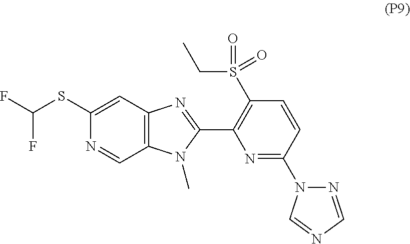 [SMILES: CCS(=O)(=O)c1ccc(-n2cncn2)nc1-c1nc2cc(SC(F)F)ncc2n1C]

Sodium chlorodifluoroacetate (642 mg, 4.18 mmol, 2.00 equiv.) was added portionwise to a mixture of potassium carbonate (1.83 g, 12.6 mmol, 6.00 equiv.), tris(2-carboxyethyl)phosphine hydrochloride (918 mg, 3.14 mmol, 1.50 equiv.) and 2-[3-ethylsulfonyl-6-(1,2,4-triazol-1-yl)-2-pyridyl]-3-methyl-imidazo[4,5-c]pyridine-6-thiol (compound 112 prepared above, 840 mg, 2.09 mmol, 1.00 equiv.) in N,N-dimethylformamide (11 mL) under argon. The reaction mixture was heated to 90° C. and stirred overnight. After cooling down to room temperature, the reaction was quenched with water, and the aqueous phase was extracted twice with ethyl acetate. The combined organic phases were washed four times with water, dried over sodium sulfate, filtered and evaporated. Purification of the crude material by flash chromatography over silica gel, eluting with methanol in dichloromethane, followed by a second purification via reverse phase flash chromatography, afforded the desired product. LCMS (method 1): 452 (M+H)⁺; retention time: 0.86 min.

### Step 8: Preparation of 6-(difluoromethylsulfonyl)-2-[3-ethylsulfonyl-6-(1,2,4-triazol-1-yl)-2-pyridyl]-3-methyl-imidazo[4,5-c]pyridine (Compound P7)

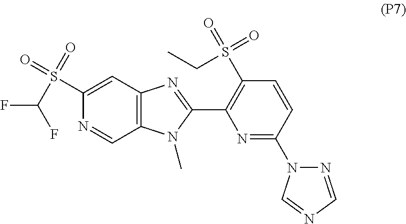 [SMILES: CCS(=O)(=O)c1ccc(-n2cncn2)nc1-c1nc2cc(S(=O)(=O)C(F)F)ncc2n1C]

Sodium periodate (398 mg, 1.83 mmol, 4.60 equiv.) followed by trichlororuthenium hydrate (6 mg, 0.01 mmol, 0.0250 equiv.) were added to a suspension of 6-(difluoromethylsulfanyl)-2-[3-ethylsulfonyl-6-(1,2,4-triazol-1-yl)-2-pyridyl]-3-methyl-imidazo[4,5-c]pyridine (compound P96, prepared as described above, 180 mg, 0.40 mmol, 1.00 equiv.) in a mixture of acetonitrile (518 μL), carbon tetrachloride (518 μL) and water (1.28 mL). The reaction mixture was stirred at room temperature for 6 hours, then diluted with water and extracted several times with dichloromethane. The combined organic phases were dried over sodium sulfate, filtered and concentrated. The crude material was purified by flash chromatography over silica gel, eluting with methanol in dichloromethane, followed by a reverse phase chromatography, to afford the desired product. LCMS (method 1): 484 (M+H)⁺; retention time: 0.77 min.

### Example P8: Preparation of 6-(difluoromethylsulfinyl)-2-[3-ethylsulfonyl-6-(1,2,4-triazol-1-yl)-2-pyridyl]-3-methyl-imidazo[4,5-c]pyridine (Compound P8)

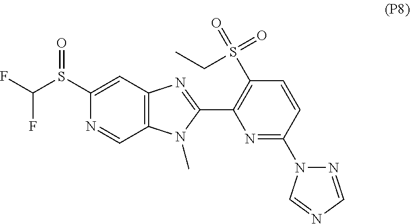 [SMILES: CCS(=O)(=O)c1ccc(-n2cncn2)nc1-c1nc2cc(S(=O)C(F)F)ncc2n1C]

3-chloroperbenzoic acid (101 mg, 0.44 mmol, 1.10 equiv.) was added portionwise to a 0° C. cooled solution of 6-(difluoromethylsulfanyl)-2-[3-ethylsulfonyl-6-(1,2,4-triazol-1-yl)-2-pyridyl]-3-methyl-imidazo[4,5-c]pyridine (compound P9, prepared as described in step 7 for Example P7, 180 mg, 0.40 mmol, 1.00 equiv.) in dichloromethane (4.0 mL). After stirring for 18 hours at room temperature, the reaction mixture was poured over a saturated sodium hydrogenocarbonate solution. The phases were separated, the organic phase washed again with a saturated sodium hydrogenocarbonate solution.

The combined aqueous phases were extracted with dichloromethane, the combined organic phases were tested for presence of peroxides then dried over sodium sulfate, filtered and concentrated.

Purification by flash chromatography over silica gel, eluting with methanol in dichloromethane, afforded the desired product as a white solid. LCMS (method 1): 468 (M+H)⁺; retention time: 0.72 min.

### Example P10: Preparation of 6-(difluoromethylsulfonyl)-2-(3-ethylsulfonyl-5-isopropoxy-2-pyridyl)-3-methyl-imidazo[4,5-c]pyridine (Compound P10)

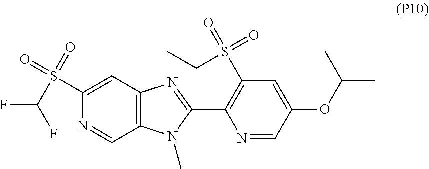 [SMILES: CCS(=O)(=O)c1cc(OC(C)C)cnc1-c1nc2cc(S(=O)(=O)C(F)F)ncc2n1C]

### Step 1: Preparation of methyl 3-ethylsulfonyl-5-(4,4,5,5-tetramethyl-1,3,2-dioxaborolan-2-yl)pyridine-2-carboxylate (compound 113)

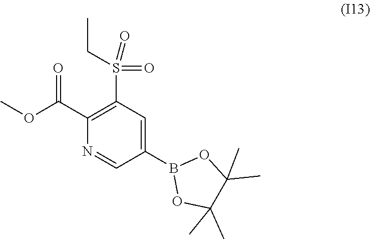

4,4,5,5-Tetramethyl-2-(4,4,5,5-tetramethyl-1,3,2-dioxaborolan-2-yl)-1,3,2-dioxaborolane (1.65 g, 6.50 mmol, 2.00 equiv.) was added to a solution of methyl 5-bromo-3-ethylsulfonyl-pyridine-2-carboxylate (prepared as described in WO 2016/087265, 1.00 g, 3.25 mmol, 1.00 equiv.) in 1,4-dioxane (9.7 mL) under argon, followed by potassium acetate (644 mg, 6.50 mmol, 2.00 equiv.), and (E)-1-chlorobut-2-ene; palladium; tricyclohexylphosphane (78 mg, 0.163 mmol, 0.05 equiv.). The reaction mixture was heated to 90° C. and stirred overnight. After cooling down to room temperature, the mixture was diluted with water, and the aqueous phase was extracted twice with ethyl acetate. The combined organic phases were washed twice with water, once with brine, dried over sodium sulfate, filtered and concentrated. The dark oil obtained was used in the next step directly. LCMS (method 1): 274 (M+H) for the corresponding boronic acid; retention time: 0.52 min (in this case, M corresponds to the corresponding boronic acid and not the pinacol ester).

### Step 2: Preparation of methyl 3-ethylsulfonyl-5-hydroxy-pyridine-2-carboxylate (compound 114)

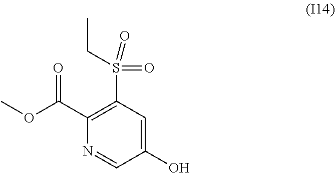 [SMILES: CCS(=O)(=O)c1cc(O)cnc1C(=O)OC]

A 30% mass hydrogen peroxide solution (4.7 ml, 46.5 mmol, 15.0 equiv.) was added to a mixture of methyl 3-ethylsulfonyl-5-(4,4,5,5-tetramethyl-1,3,2-dioxaborolan-2-yl)pyridine-2-carboxylate (compound 113 prepared above, crude, 2.20 g, 3.10 mmol) in tetrahydrofuran (19.0 ml). After stirring at room temperature overnight, the reaction mixture was diluted with water, and the aqueous phase was extracted twice with ethyl acetate. The pH of the aqueous phase was then neutralized by addition of a 2M hydrochloric acid aqueous solution, and the aqueous phase was extracted four times with dichloromethane. The combined organic phases were washed with a saturated sodium thiosulfate solution, dried over sodium sulfate, filtered and concentrated. The crude material was used in the next step directly. LCMS (method 1): 246 (M+H)⁺; retention time: 0.55 min.

### Step 3: Preparation of methyl 3-ethylsulfonyl-5-isopropoxy-pyridine-2-carboxylate (compound 114)

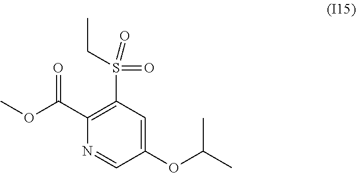 [SMILES: CCS(=O)(=O)c1cc(OC(C)C)cnc1C(=O)OC]

2-Bromopropane (197 μL, 2.09 mmol, 1.10 equiv.) was added to a mixture of methyl 3-ethylsulfonyl-5-hydroxy-pyridine-2-carboxylate (compound 114 prepared above, 467 mg, 1.90 mmol, 1.00 equiv.) and cesium carbonate (745 mg, 2.29 mmol, 1.20 equiv.) in acetonitrile (5.7 mL) under argon. The reaction mixture was heated to 70° C. and stirred overnight. After cooling down to room temperature, the reaction mixture was diluted with water, and the aqueous phase was extracted twice with ethyl acetate. The combined organic phases were dried over sodium sulfate, filtered and concentrated. The crude material was pure enough to be engaged in the next step directly. LCMS (method 1): 288 (M+H)⁺; retention time: 0.88 min.

### Step 4: Preparation of 3-ethylsulfonyl-5-isopropoxy-pyridine-2-carboxylic acid (compound 116)

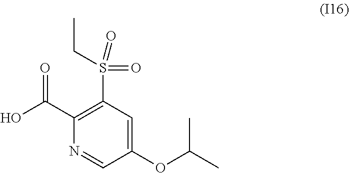 [SMILES: CCS(=O)(=O)c1cc(OC(C)C)cnc1C(=O)O]

Lithium hydroxide (33 mg, 1.37 mmol, 1.20 equiv.) was added to a solution of methyl 3-ethylsulfonyl-5-isopropoxy-pyridine-2-carboxylate (compound 115 prepared above, 328 mg, 1.14 mmol, 1.00 equiv.) in tetrahydrofuran (2.9 mL) and water (0.6 mL). After stirring overnight at room temperature, the reaction mixture was diluted with water, and the pH was acidified to 2 by adding a 2M hydrochloric acid aqueous solution. The aqueous phase was extracted with ethyl acetate, the combined organic phases were washed with water, dried over sodium sulfate, filtered and concentrated. The crude material was engaged in the next step directly. LCMS (method 1): 274 (M+H)⁺; retention time: 0.69 min.

### Step 5: Preparation of N-[2-bromo-5-(methylamino)-4-pyridyl]-3-ethylsulfonyl-5-isopropoxy-pyridine-2-carboxamide and N-(4-amino-6-bromo-3-pyridyl)-3-ethylsulfonyl-5-isopropoxy-N-methyl-pyridine-2-carboxamide (compounds 117)

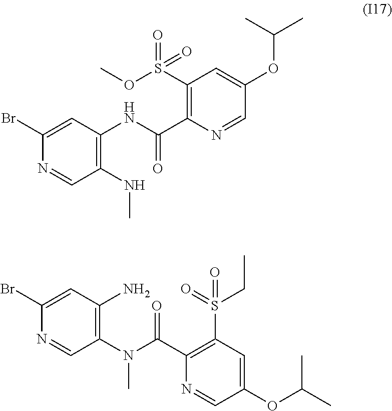 [SMILES: CNc1cnc(Br)cc1NC(=O)c1ncc(OC(C)C)cc1S(=O)(=O)OC ; CCS(=O)(=O)c1cc(OC(C)C)cnc1C(=O)N(C)c1cnc(Br)cc1N]

Oxalyl chloride (783 μL, 9.26 mmol, 1.50 equiv.) was added dropwise to a solution of 3-ethylsulfonyl-5-isopropoxy-pyridine-2-carboxylic acid (compound 116 prepared above, 2.30 g, 8.42 mmol, 1.00 equiv., in DCM (42 mL) containing a catalytic amount of N,N-dimethylformamide (33 μL, 0.42 mmol, 0.05 equiv.). The reaction mixture was stirred for 1 hour at room temperature. The dark solution was concentrated under vacuum and used directly.

A suspension of 3-ethylsulfonyl-5-isopropoxy-pyridine-2-carbonyl chloride (crude, 2.40 g, 8.20 mmol 1.10 equiv., obtained above) in tetrahydrofuran (28 mL) was added slowly to a 0° C. cooled solution of 6-bromo-N3-methyl-pyridine-3,4-diamine (1.50 g, 7.40 mmol, 1.00 equiv., prepared as described in WO 2016107831) and Hunig's base (1.90 mL, 11.0 mmol, 1.5 equiv.) in tetrahydrofuran (28 mL). The reaction mixture was stirred for 1 hour at 0° C. The mixture was then quenched with water, and the aqueous phase was extracted with ethyl acetate. The combined organic phases were washed with water, brine, dried over sodium sulfate, filtered and concentrated. The crude material was used in the next step directly without purification. LCMS (method 1): 457, 459 (M+H)⁺; retention time: 0.87 min.

### Step 6: Preparation of 6-bromo-2-(3-ethylsulfonyl-5-isopropoxy-2-pyridyl)-3-methyl-imidazo[4,5-c]pyridine (Compound 118)

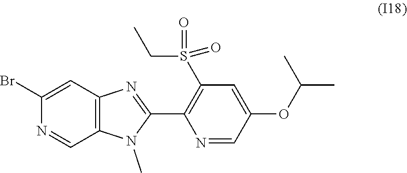 [SMILES: CCS(=O)(=O)c1cc(OC(C)C)cnc1-c1nc2cc(Br)ncc2n1C]

A solution of crude N-[2-bromo-5-(methylamino)-4-pyridyl]-3-ethylsulfonyl-5-isopropoxy-pyridine-2-carboxamide and N-(4-amino-6-bromo-3-pyridyl)-3-ethylsulfonyl-5-isopropoxy-N-methyl-pyridine-2-carboxamide (compounds 117 prepared above, 3.23 g, 7.05 mmol) in acetic acid (28 mL) was heated at 120° C. and stirred overnight. After cooling down to room temperature, the reaction mixture was poured over iced water. The aqueous phase was extracted three times with ethyl acetate, the combined organic phases were dried over sodium sulfate, filtered and concentrated. The crude material was pure enough to be used in the next step directly. LCMS (method 1): 439, 441 (M+H)⁺; retention time: 0.98 min.

### Step 7: Preparation of 2-(3-ethylsulfonyl-5-isopropoxy-2-pyridyl)-6-[(4-methoxyphenyl)methylsulfanyl]-3-methyl-imidazo[4,5-c]pyridine (Compound 119)

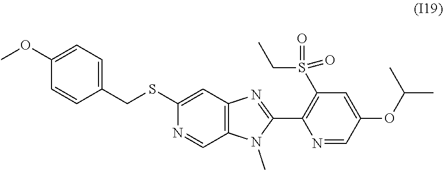 [SMILES: CCS(=O)(=O)c1cc(OC(C)C)cnc1-c1nc2cc(SCc3ccc(OC)cc3)ncc2n1C]

4-Methoxybenzyl mercaptan (979 μL, 7.06 mmol, 1.10 equiv.) was added to a mixture of 6-bromo-2-(3-ethylsulfonyl-5-isopropoxy-2-pyridyl)-3-methyl-imidazo[4,5-c]pyridine (compound 118 prepared above, crude, 2.82 g, 6.42 mmol, 1.00 equiv.), 3-chloroprop-1-ene-(5-diphenylphosphanyl-9,9-dimethyl-xanthen-4-yl)-diphenyl-phosphane; palladium (244 mg, 0.32 mmol, 0.05 equiv.) and Hunig's base (3.30 mL, 19.3 mmol, 3.00 equiv.) in 1,4-dioxane (32 mL) under argon. The reaction mixture was heated at 80° C. and stirred for 2.5 hours. After cooling down to room temperature, the mixture was diluted with water, and the aqueous phase was extracted twice with ethyl acetate. The combined organic phases were washed four times with water, dried over sodium sulfate, filtered and concentrated. The crude material was used in the next step directly. LCMS (method 1): 513 (M+H)⁺; retention time: 1.09 min.

### Step 8: Preparation of 2-(3-ethylsulfonyl-5-isopropoxy-2-pyridyl)-3-methyl-imidazo[4,5-c]pyridine-6-thiol (compound 120)

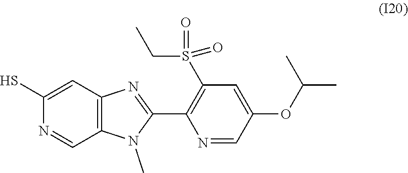 [SMILES: CCS(=O)(=O)c1cc(OC(C)C)cnc1-c1nc2cc(S)ncc2n1C]

A mixture of 2-(3-ethylsulfonyl-5-isopropoxy-2-pyridyl)-6-[(4-methoxyphenyl)methylsulfanyl]-3-methyl-imidazo[4,5-c]pyridine (compound 119 prepared above, 3.63 g, 6.55 mmol) in trifluoroacetic acid (20 mL) was heated at 75° C. and stirred overnight. After cooling down to room temperature, the reaction mixture was concentrated under vacuum. The crude material was used in the next step directly. LCMS (method 2): 394 (M+H)⁺; retention time: 0.95 min.

### Step 9: Preparation of 6-(difluoromethylsulfanyl)-2-(3-ethylsulfonyl-5-isopropoxy-2-pyridyl)-3-methyl-imidazo[4,5-c]pyridine (Compound P12)

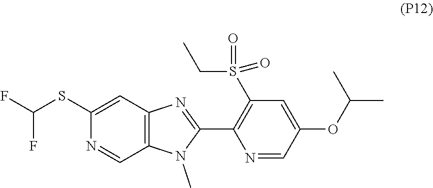 [SMILES: CCS(=O)(=O)c1cc(OC(C)C)cnc1-c1nc2cc(SC(F)F)ncc2n1C]

Sodium chlorodifluoroacetate (2.01 g, 13.1 mmol, 2.00 equiv.) was added portionwise to a mixture of potassium carbonate (5.51 g, 39.3 mmol, 6.00 equiv.), 3-[bis(2-carboxyethyl)phosphanyl]propanoic acid; hydrochloride (2.82 g, 9.82 mmol, 1.50 equiv.) and 2-(3-ethylsulfonyl-5-isopropoxy-2-pyridyl)-3-methyl-imidazo[4,5-c]pyridine-6-thiol (compound 120 prepared above, 2.57 g, 6.55 mmol, 1.00 equiv.)

in N,N-dimethylformamide (33 mL) under argon. The reaction mixture was heated to 100° C. and stirred for 2 hours. After cooling down to room temperature, the reaction was quenched with water, and the aqueous phase was extracted twice with ethyl acetate. The combined organic phases were washed four times with water, dried over sodium sulfate, filtered and evaporated. Purification of the crude material by flash chromatography over silica gel, eluting with ethyl acetate in cyclohexane, afforded the desired product. LCMS (method 1): 444 (M+H)⁺; retention time: 1.02 min.

### Step 10: Preparation of 6-(difluoromethylsulfonyl)-2-(3-ethylsulfonyl-5-isopropoxy-2-pyridyl)-3-methyl-imidazo[4,5-c]pyridine (Compound P10)

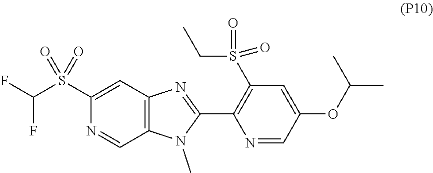 [SMILES: CCS(=O)(=O)c1cc(OC(C)C)cnc1-c1nc2cc(S(=O)(=O)C(F)F)ncc2n1C]

Sodium periodate (561 mg, 2.60 mmol, 4.60 equiv.) followed by trichlororuthenium hydrate (8 mg, 0.01 mmol, 0.0250 equiv.) were added to a suspension of 6-(difluoromethylsulfanyl)-2-(3-ethylsulfonyl-5-isopropoxy-2-pyridyl)-3-methyl-imidazo[4,5-c]pyridine (compound P12, prepared as described above, 250 mg, 0.570 mmol, 1.00 equiv.) in a mixture of acetonitrile (729 μL), carbon tetrachloride (729 μL) and water (1.82 mL). The reaction mixture was stirred at room temperature for 3 hours, then diluted with water and extracted several times with dichloromethane. The combined organic phases were washed with a saturated sodium thiosulfate solution, dried over sodium sulfate, filtered and concentrated. The crude material was purified by flash chromatography over silica gel, eluting with ethyl acetate in cyclohexane, to afford the desired product as a white solid. LCMS (method 1): 476 (M+H)⁺; retention time: 0.94 min.

### Example P11: Preparation of 6-(difluoromethylsulfinyl)-2-(3-ethylsulfonyl-5-isopropoxy-2-pyridyl)-3-methyl-imidazo[4,5-c]pyridine (Compound P11)

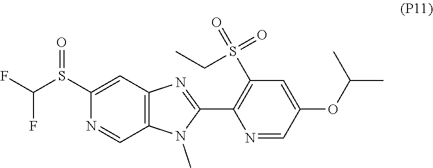 [SMILES: CCS(=O)(=O)c1cc(OC(C)C)cnc1-c1nc2cc(S(=O)C(F)F)ncc2n1C]

3-chloroperbenzoic acid (143 mg, 0.62 mmol, 1.10 equiv.) was added portionwise to a 0° C. cooled solution of 6-(difluoromethylsulfanyl)-2-(3-ethylsulfonyl-5-isopropoxy-2-pyridyl)-3-methyl-imidazo[4,5-c]pyridine (compound P12 prepared as described in step 9 for EXAMPLE P10, 250 mg, 0.56 mmol, 1.00 equiv.) in dichloromethane (2.8 mL). After stirring for 3 hours at room temperature, the reaction mixture was poured over 10% sodium thiosulfate aqueous solution. The phases were separated, the organic phase washed again with a saturated sodium hydrogenocarbonate solution, brine, dried over sodium sulfate, filtered and concentrated. Purification by flash chromatography over silica gel, eluting with ethyl acetate in cyclohexane, afforded the desired product as a white solid. LCMS (method 1): 460 (M+H)⁺; retention time: 0.88 min.

TABLE P
Examples of compounds of formula (I)

| Compound | | | RT | [M + H] | | MP |
|---|---|---|---|---|---|---|
| No. | IUPAC Name | Structure | (min) | (measured) | Method | ° C. |
| P1 | 6- (difluoromethylsul- fonyl)-2-[3- ethylsulfonyl-5- (trifluoromethyl)-2- pyridyl]-3-methyl- imidazo[4,5- c]pyridine | 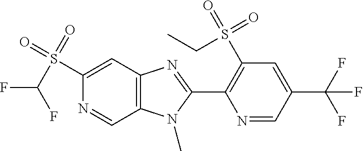 [SMILES: CCS(=O)(=O)c1cc(C(F)(F)F)cnc1-c1nc2cc(S(=O)(=O)C(F)F)ncc2n1C] | 0.95 | 485 | 1 | 169-170 |
| P2 | 6- (difluoromethylsul- finyl)-2-[3- ethylsulfonyl-5- (trifluoromethyl)-2- pyridyl]-3-methyl- imidazo[4,5- c]pyridine | 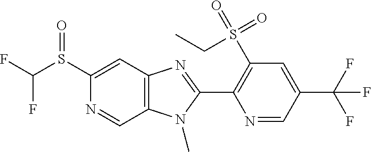 [SMILES: CCS(=O)(=O)c1cc(C(F)(F)F)cnc1-c1nc2cc(S(=O)C(F)F)ncc2n1C] | 0.89 | 469 | 1 | 175-176 |
| P3 | 6- (difluoromethylsul- fanyl)-2-[3- ethylsulfonyl-5- (trifluoromethyl)-2- pyridyl]-3-methyl- imidazo[4,5- c]pyridine | 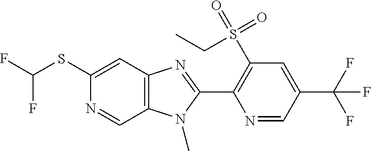 [SMILES: CCS(=O)(=O)c1cc(C(F)(F)F)cnc1-c1nc2cc(SC(F)F)ncc2n1C] | 1.01 | 453 | 1 | |
| P4 | 6- (difluoromethylsul- fonyl)-2-(3- ethylsulfonyl-2- pyridyl)-3-methyl- imidazo[4,5- c]pyridine | 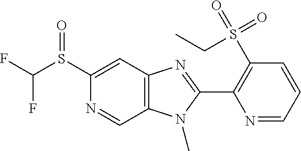 [SMILES: CCS(=O)(=O)c1cccnc1-c1nc2cc(S(=O)C(F)F)ncc2n1C] | 0.78 | 417 | 1 | 227-229 |
| P5 | 6- (difluoromethylsul- finyl)-2-(3- ethylsulfonyl-2- pyridyl)-3-methyl- imidazo[4,5- c]pyridine | 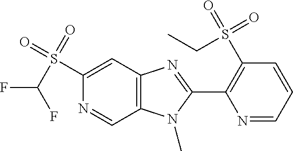 [SMILES: CCS(=O)(=O)c1cccnc1-c1nc2cc(S(=O)(=O)C(F)F)ncc2n1C] | 0.68 | 401 | 1 | 176-179 |
| P6 | 6- (difluoromethylsul- fanyl)-2-(3- ethylsulfonyl-2- pyridyl)-3-methyl- imidazo[4,5- c]pyridine | 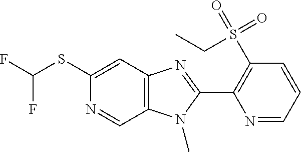 [SMILES: CCS(=O)(=O)c1cccnc1-c1nc2cc(SC(F)F)ncc2n1C] | 0.84 | 385 | 1 | 204-205 |
| P7 | 6- (difluoromethylsul- fonyl)-2-[3- ethylsulfonyl-6- (1,2,4-triazol-1-yl)- 2-pyridyl]-3- methyl- imidazo[4,5- c]pyridine | 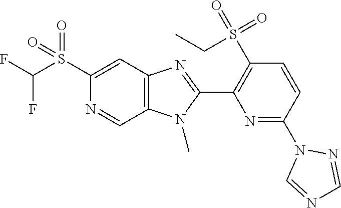 [SMILES: CCS(=O)(=O)c1ccc(-n2cncn2)nc1-c1nc2cc(S(=O)(=O)C(F)F)ncc2n1C] | 0.77 | 484 | 1 | 282-283 |
| P8 | 6- (difluoromethylsul- finyl)-2-[3- ethylsulfonyl-6- (1,2,4-triazol-1-yl)- 2-pyridyl]-3- methyl- imidazo[4,5- c]pyridine | 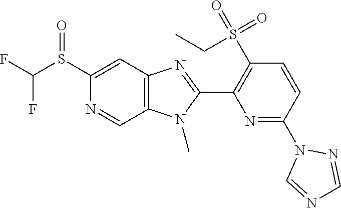 [SMILES: CCS(=O)(=O)c1ccc(-n2cncn2)nc1-c1nc2cc(S(=O)C(F)F)ncc2n1C] | 0.72 | 468 | 1 | 255-256 |
| P9 | 6- (difluoromethylsul- fanyl-2-[3- ethylsulfonyl-6- (1,2,4-triazol-1-yl)- 2-pyridyl]-3- methyl- imidazo[4,5- c]pyridine | 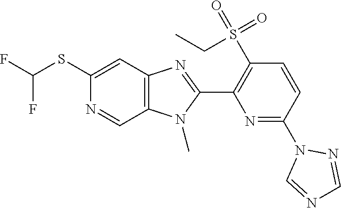 [SMILES: CCS(=O)(=O)c1ccc(-n2cncn2)nc1-c1nc2cc(SC(F)F)ncc2n1C] | 0.86 | 452 | 1 | 182-183 |
| P10 | 6- (difluoromethylsul- fonyl)-2-(3- ethylsulfonyl-5- isopropoxy-2- pyridyl)-3-methyl- imidazo[4,5- c]pyridine | 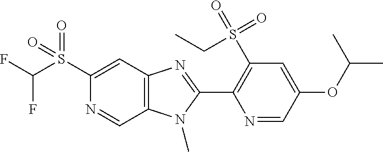 [SMILES: CCS(=O)(=O)c1cc(OC(C)C)cnc1-c1nc2cc(S(=O)(=O)C(F)F)ncc2n1C] | 0.94 | 476 | 1 | 165-167 |
| P11 | 6- (difluoromethylsul- finyl)-2-(3- ethylsulfonyl-5- isopropoxy-2- pyridyl)-3-methyl- imidazo[4,5- c]pyridine | 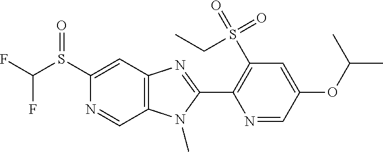 [SMILES: CCS(=O)(=O)c1cc(OC(C)C)cnc1-c1nc2cc(S(=O)C(F)F)ncc2n1C] | 0.88 | 460 | 1 | 203-205 |
| P12 | 6- (difluoromethylsul- fanyl)-2-(3- ethylsulfonyl-5- isopropoxy-2- pyridyl)-3-methyl- imidazo[4,5- c]pyridine | 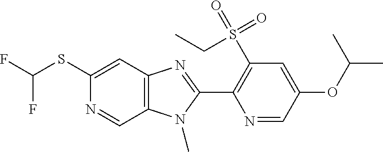 [SMILES: CCS(=O)(=O)c1cc(OC(C)C)cnc1-c1nc2cc(SC(F)F)ncc2n1C] | 1.02 | 444 | 1 | 149-151 |

TABLE I1
Examples of intermediate compounds of formula (VIII), (XIII-a) and (X):

| Compound | | | RT | [M + H] | | MP |
|---|---|---|---|---|---|---|
| No. | IUPAC Name | Structure | (min) | (measured) | Method | ° C. |
| I1 | 2-[3-ethylsulfonyl- 5-(trifluoromethyl)- 2-pyridyl]-6-[(4- methoxyphenyl) methylsulfanyl]-3- methyl- imidazo[4,5- c]pyridine | 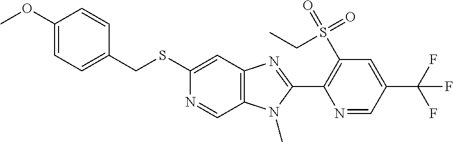 [SMILES: CCS(=O)(=O)c1cc(C(F)(F)F)cnc1-c1nc2cc(SCc3ccc(OC)cc3)ncc2n1C] | 1.08 | 523 | 1 | |
| I2 | 2-[3-ethylsulfonyl- 5-(trifluoromethyl)- 2-pyridyl]-3- methyl- imidazo[4,5- c]pyridine-6-thiol | 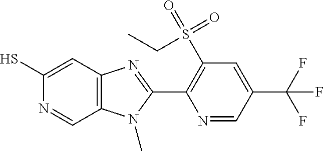 [SMILES: CCS(=O)(=O)c1cc(C(F)(F)F)cnc1-c1nc2cc(S)ncc2n1C] | 0.71 | 403 | 1 | |
| I3 | N-[2-bromo-5- (methylamino)-4- pyridyl]-3- ethylsulfanyl- pyridine-2- carboxamide and N-(4-amino-6- bromo-3-pyridyl)- 3-ethylsulfanyl-N- methyl-pyridine-2- carboxamide | 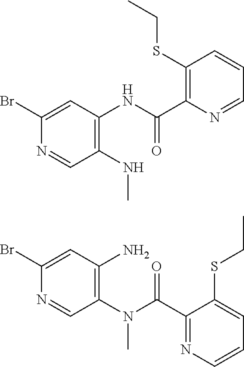 [SMILES: CCSc1cccnc1C(=O)Nc1cc(Br)ncc1NC ; CCSc1cccnc1C(=O)N(C)c1cnc(Br)cc1N] | 0.74 | 367 | 1 | |
| I4 | 6-bromo-2-(3- ethylsulfanyl-2- pyridyl)-3-methyl- imidazo[4,5- c]pyridine | 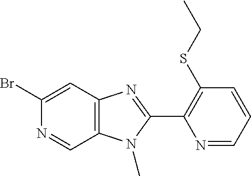 [SMILES: CCSc1cccnc1-c1nc2cc(Br)ncc2n1C] | 0.86 | 349 | 1 | |
| I5 | 6-bromo-2-(3- ethylsulfonyl-2- pyridyl)-3-methyl- imidazo[4,5- c]pyridine | 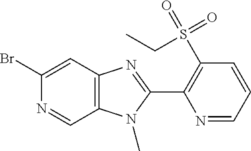 [SMILES: CCS(=O)(=O)c1cccnc1-c1nc2cc(Br)ncc2n1C] | 0.76 | 381 | 1 | |
| I6I | 6- (difluoromethylsul- fanyl)-2-(3- ethylsulfonyl-2- pyridyl)-3-methyl- imidazo[4,5- c]pyridine | 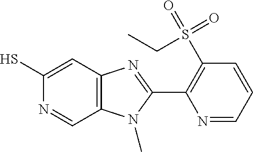 [SMILES: CCS(=O)(=O)c1cccnc1-c1nc2cc(S)ncc2n1C] | 0.29 | 335 | 1 | |
| I7 | methyl 3- ethylsulfonyl-6- (1,2,4-triazol-1- yl)pyridine-2- carboxylate | 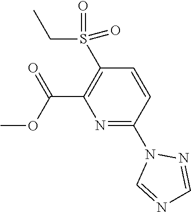 [SMILES: CCS(=O)(=O)c1ccc(-n2cncn2)nc1C(=O)OC] | 0.67 | 297 | 1 | |
| I8 | 3-ethylsulfonyl-6- (1,2,4-triazol-1- yl)pyridine-2- carboxylic acid | 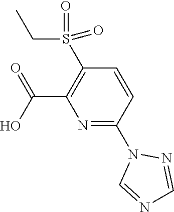 [SMILES: CCS(=O)(=O)c1ccc(-n2cncn2)nc1C(=O)O] | 0.26 | 283 | 1 | |
| I9 | N-[2-bromo-5- (methylamino)-4- pyridyl]-3- ethylsulfonyl-6- (1,2,4-triazol-1- yl)pyridine-2- carboxamide and N-(4-amino-6- bromo-3-pyridyl)- 3-ethylsulfonyl-N- methyl-6-(1,2,4- triazol-1- yl)pyridine-2- carboxamide | 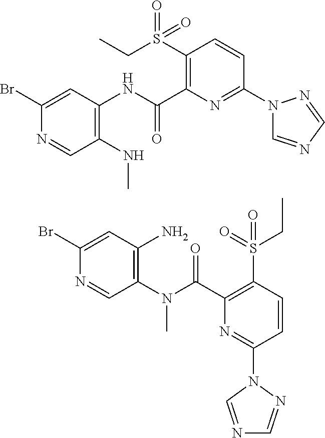 [SMILES: CCS(=O)(=O)c1ccc(-n2cncn2)nc1C(=O)Nc1cc(Br)ncc1NC ; CCS(=O)(=O)c1ccc(-n2cncn2)nc1C(=O)N(C)c1cnc(Br)cc1N] | 0.71 | 466 | 1 | |
| I10 | 6-bromo-2-[3- ethylsulfonyl-6- (1,2,4-triazol-1-yl)- 2-pyridyl]-3- methyl- imidazo[4,5- c]pyridine | 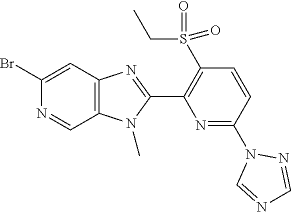 [SMILES: CCS(=O)(=O)c1ccc(-n2cncn2)nc1-c1nc2cc(Br)ncc2n1C] | 0.82 | 448 | 1 | |
| I11 | 2-[3-ethylsulfonyl- 6-(1,2,4-triazol-1- yl)-2-pyridyl]-6-[(4- methoxyphenyl) methylsulfanyl]-3- methyl- imidazo[4,5- c]pyridine | 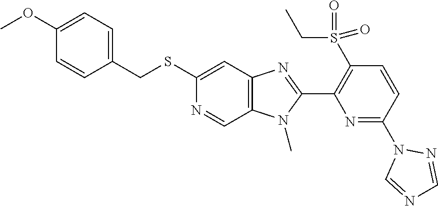 [SMILES: CCS(=O)(=O)c1ccc(-n2cncn2)nc1-c1nc2cc(SCc3ccc(OC)cc3)ncc2n1C] | 0.96 | 522 | 1 | |
| I12 | of 2-[3- ethylsulfonyl-6- (1,2,4-triazol-1-yl)- 2-pyridyl]-3- methyl- imidazo[4,5- c]pyridine-6-thiol | 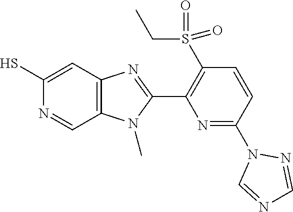 [SMILES: CCS(=O)(=O)c1ccc(-n2cncn2)nc1-c1nc2cc(S)ncc2n1C] | 0.55 | 402 | 1 | |
| I13 | methyl 3- ethylsulfonyl-5- (4,4,5,5- tetramethyl-1,3,2- dioxaborolan-2- yl)pyridine-2- carboxylate | 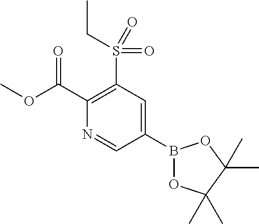 | 0.52 | 274 (boronic acid) | 1 | |
| I14 | methyl 3- ethylsulfonyl-5- hydroxy-pyridine- 2-carboxylate | 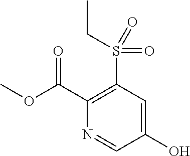 [SMILES: CCS(=O)(=O)c1cc(O)cnc1C(=O)OC] | 0.55 | 246 | 1 | |
| I15 | methyl 3- ethylsulfonyl-5- isopropoxy- pyridine-2- carboxylate | 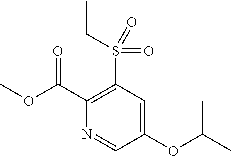 [SMILES: CCS(=O)(=O)c1cc(OC(C)C)cnc1C(=O)OC] | 0.88 | 288 | 1 | |
| I16 | 3-ethylsulfonyl-5- isopropoxy- pyridine-2- carboxylic acid | 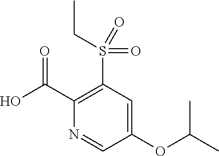 [SMILES: CCS(=O)(=O)c1cc(OC(C)C)cnc1C(=O)O] | 0.69 | 274 | 1 | |
| I17 | N-[2-bromo-5- (methylamino)-4- pyridyl]-3- ethylsulfonyl-5- isopropoxy- pyridine-2- carboxamide and N-(4-amino-6- bromo-3-pyridyl)- 3-ethylsulfonyl-5- isopropoxy-N- methyl-pyridine-2- carboxamide | 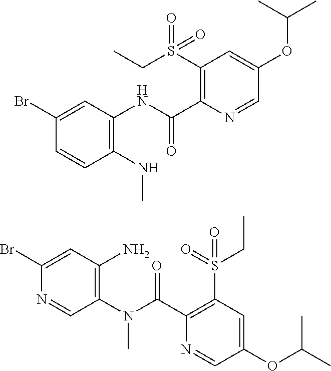 [SMILES: CCS(=O)(=O)c1cc(OC(C)C)cnc1C(=O)Nc1cc(Br)ccc1NC ; CCS(=O)(=O)c1cc(OC(C)C)cnc1C(=O)N(C)c1cnc(Br)cc1N] | 0.87 | 457 | 1 | |
| I18 | 6-bromo-2-(3- ethylsulfonyl-5- isopropoxy-2- pyridyl)-3-methyl- imidazo[4,5- c]pyridine | 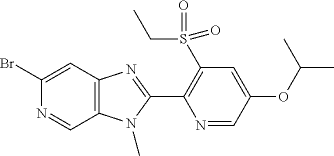 [SMILES: CCS(=O)(=O)c1cc(OC(C)C)cnc1-c1nc2cc(Br)ncc2n1C] | 0.98 | 439 | | |
| I19 | 2-(3-ethylsulfonyl- 5-isopropoxy-2- pyridyl)-6-[(4- methoxyphenyl) methylsulfanyl]-3- methyl- imidazo[4,5- c]pyridine | 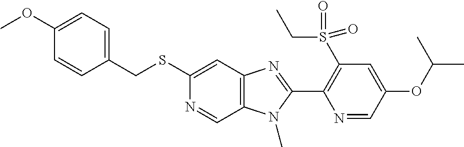 [SMILES: CCS(=O)(=O)c1cc(OC(C)C)cnc1-c1nc2cc(SCc3ccc(OC)cc3)ncc2n1C] | 1.09 | 513 | 1 | |
| I20 | 2-(3-ethylsulfonyl- 5-isopropoxy-2- pyridyl)-3-methyl- imidazo[4,5- c]pyridine-6-thiol | 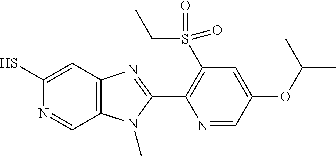 [SMILES: CCS(=O)(=O)c1cc(OC(C)C)cnc1-c1nc2cc(S)ncc2n1C] | 0.95 | 394 | 2 | |

The following mixtures of the compounds of formula I with active ingredients are preferred (the abbreviation “TX” means “one compound selected from the group consisting of the compounds described in Tables 1-9 and P of the present invention”):

an adjuvant selected from the group of substances consisting of petroleum oils (alternative name) (628)+TX,
an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chlorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos (alternative name) [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate (alternative name)+TX, bromocyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben (alternative name)+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chinomethionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin I (696)+TX, cinerin II (696)+TX, cinerins (696)+TX, clofentezine (158)+TX, closantel (alternative name) [CCN]+TX, coumaphos (174)+TX, crotamiton (alternative name) [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermethrin (201)+TX, DCPM (1032)+TX, DDT (219)+TX, demephion (1037)+TX, demephion-O (1037)+TX, demephion-S(1037)+TX, demeton (1038)+TX, demeton-methyl (224)+TX, demeton-O (1038)+TX, demeton-O-methyl (224)+TX, demeton-S(1038)+TX, demeton-S-methyl (224)+TX, demeton-S-methylsulfon (1039)+TX, diafenthiuron (226)+TX, dialifos (1042)+TX, diazinon (227)+TX, dichlofluanid (230)+TX, dichlorvos (236)+TX, dicliphos (alternative name)+TX, dicofol (242)+TX, dicrotophos (243)+TX, dienochlor (1071)+TX, dimefox (1081)+TX, dimethoate (262)+TX, dinactin (alternative name) (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram (alternative name) [CCN]+TX, disulfoton (278)+TX, DNOC (282)+TX, dofenapyn (1113)+TX, doramectin (alternative name) [CCN]+TX, endosulfan (294)+TX, endothion (1121)+TX, EPN (297)+TX, eprinomectin (alternative name) [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad (alternative name)+TX, fenpyroximate (345)+TX, fenson (1157)+TX, fentrifanil (1161)+TX, fenvalerate (349)+TX, fipronil (354)+TX, fluacrypyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (alternative name) (473)+TX, isopropyl O-(methoxyaminothiophosphoryl)salicylate (IUPAC name) (473)+TX, ivermectin (alternative name) [CCN]+TX, jasmolin I (696)+TX, jasmolin II (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen (alternative name) [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime (alternative name)

[CCN]+TX, mipafox (1293)+TX, monocrotophos (561)+TX, morphothion (1300)+TX, moxidectin (alternative name) [CCN]+TX, naled (567)+TX, NC-184 (compound code)+TX, NC-512 (compound code)+TX, nifluridide (1309)+TX, nikkomycins (alternative name) [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp′-DDT (219)+TX, parathion (615)+TX, permethrin (626)+TX, petroleum oils (alternative name) (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (alternative name) (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin I (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos (alternative name)+TX, selamectin (alternative name) [CCN]+TX, SI-0009 (compound code)+TX, sophamide (1402)+TX, spirodiclofen (738)+TX, spiromesifen (739)+TX, SSI-121 (development code) (1404)+TX, sulfiram (alternative name) [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam (alternative name)+TX, tetrachlorvinphos (777)+TX, tetradifon (786)+TX, tetranactin (alternative name) (653)+TX, tetrasul (1425)+TX, thiafenox (alternative name)+TX, thiocarboxime (1431)+TX, thiofanox (800)+TX, thiometon (801)+TX, thioquinox (1436)+TX, thuringiensin (alternative name) [CCN]+TX, triamiphos (1441)+TX, triarathene (1443)+TX, triazophos (820)+TX, triazuron (alternative name)+TX, trichlorfon (824)+TX, trifenofos (1455)+TX, trinactin (alternative name) (653)+TX, vamidothion (847)+TX, vaniliprole [CCN] and YI-5302 (compound code)+TX,

an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX,
an anthelmintic selected from the group of substances consisting of abamectin (1)+TX, crufomate (1011)+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX,
an avicide selected from the group of substances consisting of chloralose (127)+TX, endrin (1122)+TX, fenthion (346)+TX, pyridin-4-amine (IUPAC name) (23) and strychnine (745)+TX, a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX,
a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX,
a soil sterilant selected from the group of substances consisting of iodomethane (IUPAC name) (542) and methyl bromide (537)+TX,
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX,
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, gossyplure (alternative name) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B₁ (alternative name) (839)+TX, trimedlure B2 (alternative name) (839)+TX, trimedlure C (alternative name) (839) and trunc-call (alternative name) [CCN]+TX,
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX, an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 1,2-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichlorophenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethylcarbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion (alternative name) [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin (alternative name) [CCN]+TX, allyxycarb (866)+TX, alpha-cypermethrin (202)+TX, alpha-ecdysone (alternative name) [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (alternative name) (41)+TX, azamethiphos (42)+TX, azinphos-ethyl (44)+TX, azinphos-methyl (45)+TX, azothoate (889)+TX, Bacillus thuringiensis delta endotoxins (alternative name) (52)+TX, barium hexafluorosilicate (alternative name) [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrin (194)+TX, beta-cypermethrin (203)+TX, bifenthrin (76)+TX, bioallethrin (78)+TX, bioallethrin S-cyclopentenyl isomer (alternative name) (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate (alternative name)+TX, bromfenvinfos (914)+TX, bromocyclen (918)+TX, bromo-DDT (alternative name) [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben (alternative name)+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (alternative name) (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, chloroform [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin I (696)+TX, cinerin II (696)+TX, cinerins (696)+TX, cis-resmethrin (alternative name)+TX, cismethrin (80)+TX, clocythrin (alternative name)+TX, cloethocarb (999)+TX, closantel (alternative name) [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton (alternative name) [CCN]+TX, crotoxyphos (1010)+TX, crufomate (1011)+TX, cryolite (alternative name) (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate (alternative name) [CCN]+TX, d-limonene (alternative name) [CCN]+TX, d-tetramethrin (alternative name) (788)+TX, DAEP (1031)+TX, dazomet (216)+TX, DDT (219)+TX, decarbofuran (1034)+TX, deltamethrin (223)+TX, demephion (1037)+TX, demephion-O (1037)+TX, demephion-S(1037)+TX, demeton (1038)+TX, demeton-methyl (224)+TX, demeton-O (1038)+TX, demeton-O-methyl (224)+TX, demeton-S(1038)+TX, demeton-S-methyl (224)+TX, demeton-S-methylsulphon (1039)+TX, diafenthiuron (226)+TX, dialifos (1042)+TX, diamidafos (1044)+TX, diazinon (227)+TX, dicapthon (1050)+TX, dichlofenthion (1051)+TX, dichlorvos (236)+TX, dicliphos (alternative name)+TX, dicresyl (alternative name) [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor (alternative name) [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin (alternative name) [CCN]+TX, DSP (1115)+TX, ecdysterone (alternative name) [CCN]+TX, EI 1642 (development code) (1118)+TX, emamectin (291)+TX, emamectin benzoate (291)+TX, EMPC (1120)+TX, empenthrin (292)+TX, endosulfan (294)+TX, endothion (1121)+TX, endrin (1122)+TX, EPBP (1123)+TX, EPN (297)+TX, epofenonane (1124)+TX, eprinomectin (alternative name) [CCN]+TX, esfenvalerate (302)+TX, etaphos (alternative name) [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (alternative name) (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad (alternative name)+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (alternative name) (473)+TX, isodrin (1235)+TX, isofenphos (1236)+TX, isolane (1237)+TX, isoprocarb (472)+TX, isopropyl O-(methoxy-aminothiophosphoryl)salicylate (IUPAC name) (473)+TX, isoprothiolane (474)+TX, isothioate (1244)+TX, isoxathion (480)+TX, ivermectin (alternative name) [CCN]+TX, jasmolin I (696)+TX, jasmolin II (696)+TX, jodfenphos (1248)+TX, juvenile hormone I (alternative name) [CCN]+

TX, juvenile hormone II (alternative name) [CCN]+TX, juvenile hormone III (alternative name) [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (alternative name) (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (alternative name) (533)+TX, methoxychlor (534)+TX, methoxyfenozide (535)+TX, methyl bromide (537)+TX, methyl isothiocyanate (543)+TX, methylchloroform (alternative name) [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime (alternative name) [CCN]+TX, mipafox (1293)+TX, mirex (1294)+TX, monocrotophos (561)+TX, morphothion (1300)+TX, moxidectin (alternative name) [CCN]+TX, naftalofos (alternative name) [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, O-5-dichloro-4-iodophenyl O-ethyl ethylphosphonothioate (IUPAC name) (1057)+TX, 0,0-diethyl O-4-methyl-2-oxo-2H-chromen-7-yl phosphorothioate (IUPAC name) (1074)+TX, 0,0-diethyl O-6-methyl-2-propylpyrimidin-4-yl phosphorothioate (IUPAC name) (1075)+TX, 0,0,0′,0′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron (alternative name) [CCN]+TX, pentachlorophenol (623)+TX, pentachlorophenyl laurate (IUPAC name) (623)+TX, permethrin (626)+TX, petroleum oils (alternative name) (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, polychloroterpenes (traditional name) (1347)+TX, potassium arsenite [CCN]+TX, potassium thiocyanate [CCN]+TX, prallethrin (655)+TX, precocene I (alternative name) [CCN]+TX, precocene II (alternative name) [CCN]+TX, precocene Ill (alternative name) [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia (alternative name) [CCN]+TX, quinalphos (711)+TX, quinalphos-methyl (1376)+TX, quinothion (1380)+TX, quintiofos (1381)+TX, R-1492 (development code) (1382)+TX, rafoxanide (alternative name) [CCN]+TX, resmethrin (719)+TX, rotenone (722)+TX, RU 15525 (development code) (723)+TX, RU 25475 (development code) (1386)+TX, ryania (alternative name) (1387)+TX, ryanodine (traditional name) (1387)+TX, sabadilla (alternative name) (725)+TX, schradan (1389)+TX, sebufos (alternative name)+TX, selamectin (alternative name) [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (alternative name) (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam (alternative name)+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox (alternative name)+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin (alternative name) [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron (alternative name)+TX, trichlorfon (824)+TX, trichlormetaphos-3 (alternative name) [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (alternative name) (725)+TX, veratrine (alternative name) (725)+TX, XMC (853)+TX, xylylcarb (854)+TX, YI-5302 (compound code)+TX, zeta-cypermethrin (205)+TX, zetamethrin (alternative name)+TX, zinc phosphide (640)+TX, zolaprofos (1469) and ZXI 8901 (development code) (858)+TX, cyantraniliprole [736994-63-19+TX, chlorantraniliprole [500008-45-7]+TX, cyenopyrafen [560121-52-0]+TX, cyflumetofen [400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole [704886-18-0]+TX, meperfluthrin [915288-13-0]+TX, tetramethylfluthrin [84937-88-2]+TX, triflumezopyrim (disclosed in WO 2012/092115)+TX, fluxametamide (WO 2007/026965)+TX, epsilon-metofluthrin [240494-71-7]+TX, epsilon-momfluorothrin [1065124-65-3]+TX, fluazaindolizine [1254304-22-7]+TX, chloroprallethrin [399572-87-3]+TX, fluxametamide [928783-29-3]+TX, cyhalodiamide [1262605-53-7]+TX, tioxazafen [330459-31-9]+TX, broflanilide [1207727-04-5]+TX, flufiprole [704886-18-0]+TX, cyclaniliprole [1031756-98-5]+TX, tetraniliprole [1229654-66-3]+TX, guadipyr (described in WO2010/060231)+TX, cycloxaprid (described in WO 2005/077934)+TX,

a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX,
a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX,
a nitrification inhibitor selected from the group of substances consisting of potassium ethylxanthate [CCN] and nitrapyrin (580)+TX,
a plant activator selected from the group of substances consisting of acibenzolar (6)+TX, acibenzolar-S-methyl (6)+TX, probenazole (658) and Reynoutria sachalinensis extract (alternative name) (720)+TX,
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX,
a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX,
a virucide selected from the group of substances consisting of imanin (alternative name) [CCN] and ribavirin (alternative name) [CCN]+TX,
a wound protectant selected from the group of substances consisting of mercuric oxide (512)+TX, octhilinone (590) and thiophanate-methyl (802)+TX,
and biologically active compounds selected from the group consisting of azaconazole (60207-31-0]+TX, bitertanol [70585-36-3]+TX, bromuconazole [116255-48-2]+TX, cyproconazole [94361-06-5]+TX, difenoconazole [119446-68-3]+TX, diniconazole [83657-24-3]+TX, epoxiconazole [106325-08-0]+TX, fenbuconazole [114369-43-6]+TX, fluquinconazole [136426-54-5]+TX, flusilazole [85509-19-9]+TX, flutriafol [76674-21-0]+TX, hexaconazole [79983-71-4]+TX, imazalil [35554-44-0]+TX, imibenconazole [86598-92-7]+TX, ipconazole [125225-28-7]+TX, metconazole [125116-23-6]+TX, myclobutanil [88671-89-0]+TX, pefurazoate [101903-30-4]+TX, penconazole [66246-88-6]+TX, prothioconazole [178928-70-6]+TX, pyrifenox [88283-41-4]+TX, prochloraz [67747-09-5]+TX, propiconazole [60207-90-1]+TX, simeconazole [149508-90-7]+TX, tebuconazole [107534-96-3]+TX, tetraconazole [112281-77-3]+TX, triadimefon [43121-43-3]+TX, triadimenol [55219-65-3]+TX, triflumizole [99387-89-0]+TX, triticonazole [131983-72-7]+TX, ancymidol [12771-68-5]+TX, fenarimol [60168-88-9]+TX, nuarimol [63284-71-9]+TX, bupirimate [41483-43-6]+TX, dimethirimol [5221-53-4]+TX, ethirimol [23947-60-6]+TX, dodemorph [1593-77-7]+TX, fenpropidine [67306-00-7]+TX, fenpropimorph [67564-91-4]+TX, spiroxamine [118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl [71626-11-4]+TX, furalaxyl [57646-30-7]+TX, metalaxyl [57837-19-1]+TX, R-metalaxyl [70630-17-0]+TX, ofurace [58810-48-3]+TX, oxadixyl [77732-09-3]+TX, benomyl [17804-35-2]+TX, carbendazim [10605-21-7]+TX, debacarb [62732-91-6]+TX, fuberidazole [3878-19-1]+TX, thiabendazole [148-79-8]+TX, chlozolinate [84332-86-5]+TX, dichlozoline [24201-58-9]+TX, iprodione [36734-19-7]+TX, myclozoline [54864-61-8]+TX, procymidone [32809-16-8]+TX, vinclozoline [50471-44-8]+TX, boscalid [188425-85-6]+TX, carboxin [5234-68-4]+TX, fenfuram [24691-80-3]+TX, flutolanil [66332-96-5]+TX, mepronil [55814-41-0]+TX, oxycarboxin [5259-88-1]+TX, penthiopyrad [183675-82-3]+TX, thifluzamide [130000-40-7]+TX, guazatine [108173-90-6]+TX, dodine [2439-10-3][112-65-2](free base)+TX, iminoctadine [13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl [143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol [2425-06-1]+TX, captan [133-06-2]+TX, dichlofluanid [1085-98-9]+TX, fluoroimide [41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper [53988-93-5]+TX, oxine-copper [10380-28-6]+TX, dinocap [131-72-6]+TX, nitrothal-isopropyl [10552-74-6]+TX, edifenphos [17109-49-8]+TX, iprobenphos [26087-47-8]+TX, isoprothiolane [50512-35-1]+TX, phosdiphen [36519-00-3]+TX, pyrazophos [13457-18-6]+TX, tolclofos-methyl [57018-04-9]+TX, acibenzo-lar-S-methyl [135158-54-2]+TX, anilazine [101-05-3]+TX, benthiavalicarb [413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone [117-80-6]+TX, diclocymet [139920-32-4]+TX, diclomezine [62865-36-5]+TX, dicloran [99-30-9]+TX, diethofencarb [87130-20-9]+TX, dimethomorph [110488-70-5]+TX, SYP-L190 (Flumorph) [211867-47-9]+TX, dithianon [3347-22-6]+TX, ethaboxam [162650-77-3]+TX, etridiazole [2593-15-9]+TX, famoxadone [131807-57-3]+TX, fenamidone [161326-34-7]+TX, fenoxanil [115852-48-7]+TX, fentin [668-34-8]+TX, ferimzone [89269-64-7]+TX, fluazinam [79622-59-6]+TX, fluopicolide [239110-15-7]+TX, flusulfamide [106917-52-6]+TX, fenhexamid [126833-17-8]+TX, fosetyl-aluminium [39148-24-8]+TX, hymexazol [10004-44-1]+TX, iprovalicarb [140923-17-7]+TX, IKF-916 (Cyazofamid) [120116-88-3]+TX, kasugamycin [6980-18-3]+TX, methasulfocarb [66952-49-6]+TX, metrafenone [220899-03-6]+TX, pencycuron [66063-05-6]+TX, phthalide [27355-22-2]+TX, polyoxins [11113-80-7]+TX, probenazole [27605-76-1]+TX, propamocarb [25606-41-1]+TX, proquinazid [189278-12-4]+TX, pyroquilon [57369-32-1]+TX, quinoxyfen [124495-18-7]+TX, quintozene [82-68-8]+TX, sulfur [7704-34-9]+TX, tiadinil [223580-51-6]+TX, triazoxide [72459-58-6]+TX, tricyclazole [41814-78-2]+TX, triforine [26644-46-2]+TX, validamycin [37248-47-8]+TX, zoxamide (RH7281) [156052-68-5]+TX, mandipropamid [374726-62-2]+TX, isopyrazam [881685-58-1]+TX, sedaxane [874967-67-6]+TX, 3-difluoromethyl-1-methyl-1H-pyrazole-4-carboxylic acid (9-dichloromethylene-1,2,3,4-tetrahydro-1,4-methano-naphthalen-5-yl)-amide (disclosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11Hnaphtho[2,1-b]pyrano[3,4-e]pyran-4-yl]methyl-cyclopropanecarboxylate [915972-17-7]+TX and 1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide [926914-55-8]+TX; lancotrione [1486617-21-3]+TX, florpyrauxifen [943832-81-3]+TX, ipfentrifluconazole[1417782-08-1]+TX, mefentrifluconazole [1417782-03-6]+TX, quinofumelin [861647-84-9]+TX, chloroprallethrin [399572-87-3]+TX, cyhalodiamide [1262605-53-7]TX, fluazaindolizine [1254304-22-7]+TX, fluxametamide [928783-29-3]+TX, epsilon-metofluthrin [240494-71-7]+TX, epsilon-momfluorothrin [1065124-65-3]+TX, pydiflumetofen [1228284-64-7]+TX, kappa-bifenthrin [439680-76-9]+TX, broflanilide [1207727-04-5]+TX, dicloromezotiaz [1263629-39-5]+TX, dipymetitrone [16114-35-5]+TX, pyraziflumid [942515-63-1]+TX, kappa-tefluthrin [391634-71-2]+TX; fenpicoxamid [517875-34-2]+TX; fluindapyr [1383809-87-7]+TX; alpha-bromadiolone [28772-56-7]+TX; flupyrimin [1689566-03-7]+TX; benzpyrimoxan [1449021-97-9]+TX; acynonapyr [1332838-17-1]+TX; inpyrfluxam [1352994-67-2]+TX, isoflucypram [1255734-28-1]+TX; rescalure [64309-03-1]+TX; aminopyrifen [1531626-08-0]+TX; tyclopyrazoflor [1477919-27-9]+TX; and spiropidion [1229023-00-0]+TX; and microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ100)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain I-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPe1C))+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HC®+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus; and
Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame peppermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®); and
pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC—LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®+TX, (E+TX,Z+TX,Z)-3+TX,8+TX,11 Tetradecatrienyl acetate+TX, (Z+TX,Z+TX,E)-7+TX,11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate; and
Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius califomicus (Amblyline®+TX, Spical®)+TX, Amblyseius cucumeris (Thripex®+TX, Bugline Cucumeris®)+TX, Amblyseius fallacis (Fallacis®)+TX, Amblyseius swirskii (Bugline Swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, EnStrip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus califomicus+TX, Eretmocerus eremicus (Ercal®+TX, Eretline E®)+TX, Eretmocerus eremicus (Bemimix®)+TX, Eretmocerus hayati+TX, Eretmocerus mundus (Bemipar®+TX, Eretline M®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline Hm®+TX, Larvanem-M®)+TX, Hippodamia convergens+TX, Hypoaspis aculeifer (Aculeifer-System®+TX, Entomite-A®)+TX, Hypoaspis miles (Hypoline M®+TX, Entomite-M®)+TX, Lbalia leucospoides+TX, Lecanoideus floccissimus+TX, Lemophagus errabundus+TX, Leptomastidea abnormis+TX, Leptomastix dactylopfi (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline C®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEXC))+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline I®)+TX, Orius laevigatus (Thripor-L®+TX, Oriline I®)+TX, Orius majusculus (Oriline M®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline P®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline Sf®+TX, Scia-Rid®+TX, Entonem®)+TX, Steinernema kraussei (Nemasys L®+TX, BioNem L®+TX, Exhibitline Srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine B®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator; and
other biologicals including: abscisic acid+TX, bioSea®+TX, Chondrostereum purpureum (Chontrol Paste®)+TX, Colletotrichum gloeosporioides (Callego®)+TX, Copper Octanoate (Cueva®)+TX, Delta traps (Trapline D®)+TX, Erwinia amylovora (Harpin) (ProAct®+TX, Ni-HIBIT Gold CST®)+TX, Ferri-phosphate (Ferramol®)+TX, Funnel traps (Trapline Y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline F®)+TX, Microctonus hyperodae+TX, Mycoleptodiscus terrestris (Des-X®)+TX, BioGain®+TX, Aminomite®+TX, Zenox®+TX, Pheromone trap (Thripline Ams®)+TX, potassium bicarbonate (MilStop®)+TX, potassium salts of fatty acids (Sanova®)+TX, potassium silicate solution (SD-Matrix®+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+B®)+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “develoment code” is used or, if neither one of those designations nor a “common name” is used, an “alternative name” is employed. “CAS Reg. No” means the Chemical Abstracts Registry Number.

The active ingredient mixture of the compounds of formula I selected from Tables 1-9 with active ingredients described above comprises a compound selected from Tables 1-9 and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula I selected from Tables 1-9 and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables 1-9 and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

### BIOLOGICAL EXAMPLES

### Example B1: Spodoptera littoralis (Egyptian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm: P1, P2, P3, P9, P10, P11 and P12.

### Example B2: Spodoptera littoralis (Egyptian Cotton Leaf Worm)

Test compounds were applied by pipette from 10′000 ppm DMSO stock solutions into 24-well plates and mixed with agar. Lettuce seeds were placed onto the agar and the multi well plate was closed by another plate which contained also agar. After 7 days the compound was absorbed by the roots and the lettuce grew into the lid plate. The lettuce leaves were then cut off into the lid plate. Spodoptera eggs were pipetted through a plastic stencil onto a humid gel blotting paper and the lid plate was closed with it. The samples were assessed for mortality, anti-feedant effect and growth inhibition in comparison to untreated samples 6 days after infestation.

### Example B3: Plutella xylostella (Diamond Back Moth)

24-well microtiter plates with artificial diet were treated with aqueous test solutions prepared from 10′000 ppm DMSO stock solutions by pipetting. After drying, the plates were infested with L2 larvae (10 to 15 per well). The samples were assessed for mortality and growth inhibition in comparison to untreated samples 5 days after infestation.

The following compounds gave an effect of at least 80% in at least one of the two categories (mortality or growth inhibition) at an application rate of 200 ppm: P1, P2, P3, P10, P11 and P12.

### Example B4: Diabrotica balteata (Corn Root Worm)

Maize sprouts placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm: P1, P2, P3, P4, P5, P6, P7, P8, P9 and P12.

### Example B5: Myzus persicae (Green Peach Aphid)

Sunflower leaf discs were placed onto agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm: P1, P2, P3, P4, P5, P6, P7, P8, P9, P10, P11 and P12.

### Example B6: Myzus persicae (Green Peach Aphid)

Roots of pea seedlings infested with an aphid population of mixed ages were placed directly into aqueous test solutions prepared from 10′000 DMSO stock solutions. The samples were assessed for mortality 6 days after placing seedlings into test solutions.

The following compounds resulted in at least 80% mortality at a test rate of 24 ppm: P1, P2, P3, P4, P5, P6, P7, P8, P9, P10, P11 and P12.

### Example B7: Myzus persicae (Green Peach Aphid)

Test compounds prepared from 10′000 ppm DMSO stock solutions were applied by pipette into 24-well microtiter plates and mixed with sucrose solution. The plates were closed with a stretched Parafilm. A plastic stencil with 24 holes was placed onto the plate and infested pea seedlings were placed directly on the Parafilm. The infested plate was closed with a gel blotting paper and another plastic stencil and then turned upside down. The samples were assessed for mortality 5 days after infestation.

### Example B8: Bemisia tabaci (Cotton White Fly)

Cotton leaf discs were placed on agar in 24-well microtiter plates and sprayed with aqueous test solutions prepared from 10′000 ppm DMSO stock solutions. After drying the leaf discs were infested with adult white flies. The samples were checked for mortality 6 days after incubation.

The following compounds resulted in at least 80% mortality at an application rate of 200 ppm: P1, P2, P3, P8 and P9.

### Example B9: Euschistus heros (Neotropical Brown Stink Bug)

Soybean leaves on agar in 24-well microtiter plates were sprayed with aqueous test solutions prepared from 10′000 ppm DMSO stock solutions. After drying the leaves were infested with N2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm: P1, P2, P3, P4, P5, P6, P7, P8, P9, P10, P11 and P12.

### Example B10: Tetranychus urticae (Two-Spotted Spider Mite)

Bean leaf discs on agar in 24-well microtiter plates were sprayed with aqueous test solutions prepared from 10′000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm: P10 and P11.

### Example B11: Thrips tabaci (Onion Thrips)

Sunflower leaf discs were placed on agar in 24-well microtiter plates and sprayed with aqueous test solutions prepared from 10′000 ppm DMSO stock solutions. After drying the leaf discs were infested with a thrips population of mixed ages. The samples were assessed for mortality 6 days after infestation.

### Example B12: Frankliniella occidentalis (Western Flower Thrips)

Sunflower leaf discs were placed on agar in 24-well microtiter plates and sprayed with aqueous test solutions prepared from 10′000 DMSO stock solutions. After drying the leaf discs were infested with a Frankliniella population of mixed ages. The samples were assessed for mortality 7 days after infestation.

The following compound resulted in at least 80% mortality at an application rate of 200 ppm: P3.

1. A compound of formula I, 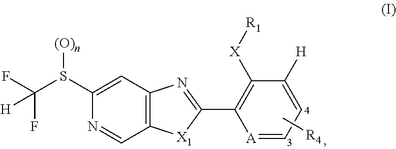
wherein
A is CH or N;
X is S, SO or SO₂;
R₁ is C₁-C₄alkyl, C₁-C₄haloalkyl or C₃-C₆cycloalkylC₁-C₄alkyl;
n is 0, 1 or 2;
X₁ is O, S or NR³, wherein R₃ is C₁-C₄alkyl; and
R₄ is hydrogen, halogen, C₁-C₆alkyl, C₁-C₆haloalkyl, C₁-C₆haloalkoxy, C₃-C₆cycloalkyl, C₃-C₆cycloalkyl-C₁-C₄alkyl, C₁-C₆cyanoalkyl, C₁-C₆alkoxy, C₁-C₆cyanoalkoxy, C₃-C₆cycloalkyl-C₁-C₄alkoxy or C₃-C₆halocycloalkyl-C₁-C₄alkoxy; or
R₄ is C₃-C₆cycloalkyl mono- or polysubstituted by substituents selected from the group consisting of halogen, cyano, C₁-C₄alkyl, C₁-C₄haloalkyl and C₃-C₆cycloalkyl; or
R₄ is a five- to ten-membered monocyclic or fused bicyclic heterocyclic ring system which can be aromatic, partially saturated or fully saturated and which contains 1 to 4 hetero atoms selected from the group consisting of nitrogen, oxygen and sulfur, with the proviso that each ring system cannot contain more than 2 oxygen atoms and not more than 2 sulfur atoms, said five- to ten-membered ring system can be mono- to polysubstituted by substituents independently selected from the group consisting of halogen, cyano, oxo, OH, NH₂, C₁-C₆alkyl, C₃-C₆cycloalkyl, C₁-C₆haloalkyl, C₁-C₆alkoxy, C₁-C₆alkylthio, and C₁-C₆alkylsulfonyl; and agrochemically acceptable salts, stereoisomers, enantiomers, tautomers and N-oxides of the compounds of formula I.

2. A compound of formula I according to claim 1, wherein
R₄ is a five- to ten-membered monocyclic or fused bicyclic heterocyclic ring system which can be aromatic, partially saturated or fully saturated and which contains 1 to 4 hetero atoms selected from the group consisting of nitrogen, oxygen and sulfur, with the proviso that each ring system cannot contain more than 2 oxygen atoms and not more than 2 sulfur atoms, said five- to ten-membered ring system can be mono- to polysubstituted by substituents independently selected from the group consisting of halogen, cyano, oxo, OH, NH₂, C₁-C₆alkyl, C₃-C₆cycloalkyl, C₁-C₆haloalkyl, C₁-C₆alkoxy, C₁-C₆alkylthio, and C₁-C₆alkylsulfonyl, pyrimidinyl, thiazolyl, pyridinyl, pyrazinyl, pyridazinyl, pyrazinyloxy, imidazolyl, dihydro-oxazolyl, oxazolyl, dioxolanyl, 1,3-dioxanyl and 5,6-dihydro-[1,4,2]dioxazinyl; wherein said pyrimidinyl, thiazolyl, pyridinyl, pyrazinyl, pyridazinyl, pyrazinyloxy, imidazolyl, dihydro-oxazolyl, oxazolyl, dioxolanyl, 1,3-dioxanyl and 5,6-dihydro-[1,4,2]dioxazinyl which itself can be mono- to polysubstituted by substituents independently selected from the group consisting of halogen, cyano, oxo, OH, NH₂, C₁-C₆alkyl, C₃-C₆cycloalkyl, C₁-C₆haloalkyl, C₁-C₆alkoxy, C₁-C₆alkylthio and C₁-C₆alkylsulfonyl.

3. A compound of formula I according to claim 1, wherein R₄ is selected from the group consisting of J-1 to J-56 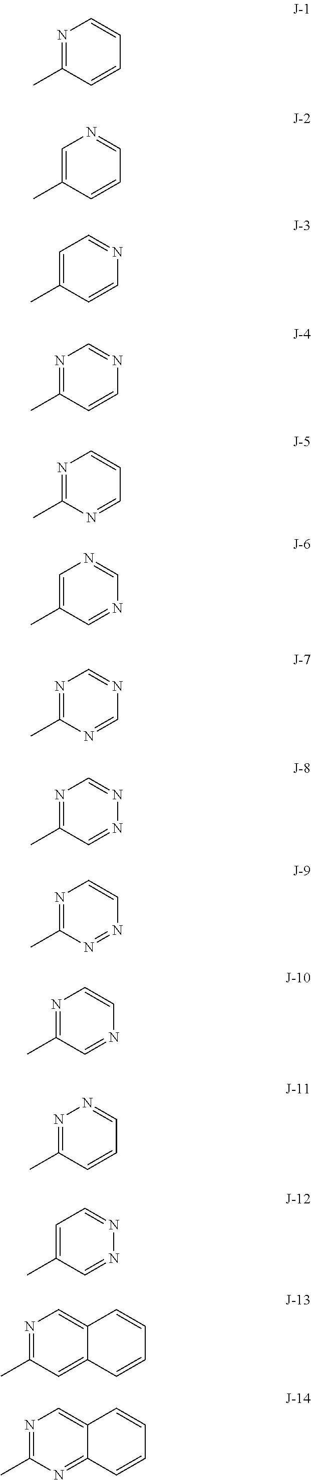 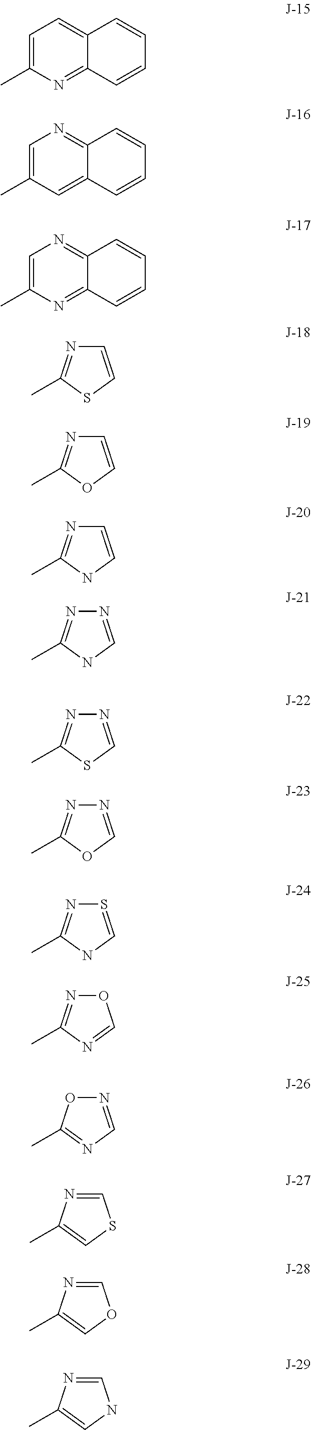 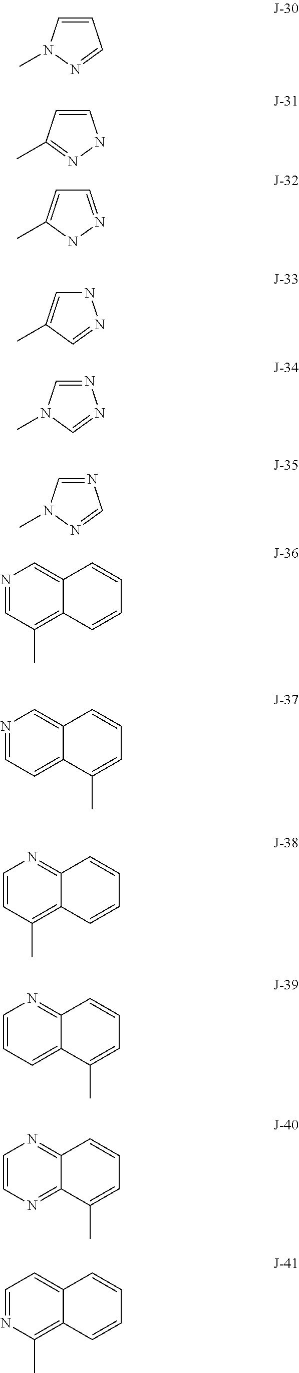 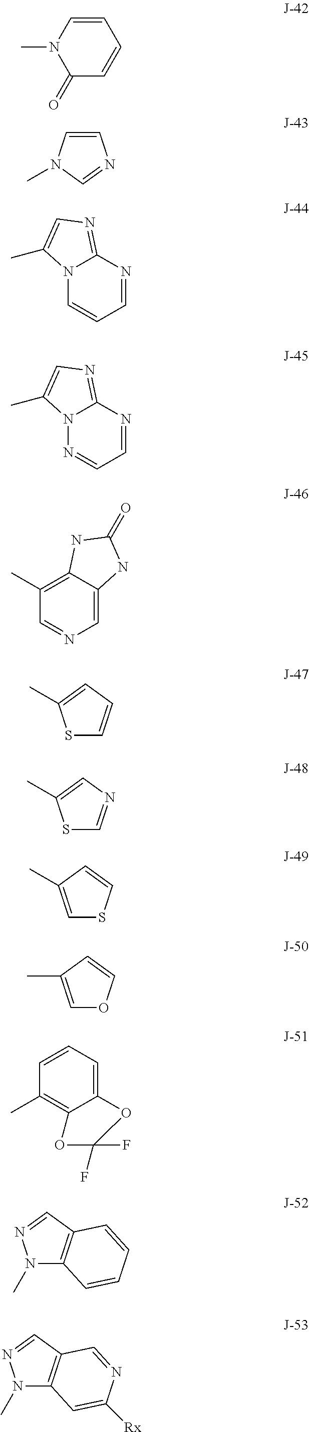 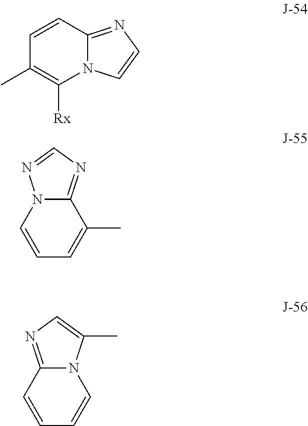
wherein each group J-1 to J-56 is mono- di- or trisubstituted with Rx, wherein
each Rx is, independently selected from the group consisting of halogen, cyano, oxo, OH, NH₂, C₁-C₆alkyl, C₃-C₆cycloalkyl, C₁-C₆haloalkyl, C₁-C₆alkoxy, C₁-C₆alkylthio and C₁-C₆alkylsulfonyl.

4. A compound of formula I according to claim 1, represented by the compounds of formula I-1 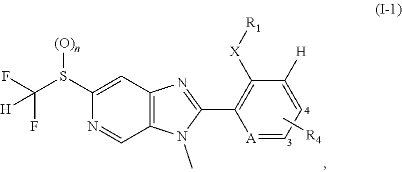
wherein A, X, R₁, R₄, and n are as defined under formula I in claim 1.

5. A compound of formula I according to claim 1, represented by the compounds of formula I-2 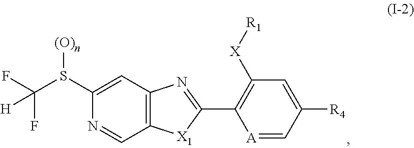
wherein A, X, X₁, R₁, R₄ and n are as defined under formula I in claim 1.

6. A compound of formula I according to claim 1, represented by the compounds of formula I-3 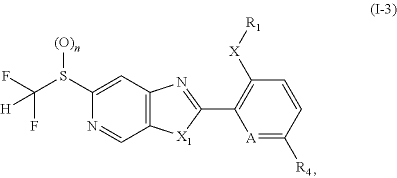
wherein A, X, X₁, R₁, R₄ and n are as defined under formula I in claim 1.

7. A compound of formula I according to claim 1, wherein
R₁ is C₁-C₄alkyl;
X₁═NR³, wherein R³ is methyl;
A is CH or N;
X is S or SO₂; and
R₄ is hydrogen, C₁-C₆haloalkyl, C₁-C₆haloalkoxy, C₁-C₆alkoxy, C₁-C₆cyanoalkoxy, C₃-C₆cycloalkyl-C₁-C₄alkoxy, 1,2,4-triazol-1-yl, pyrazol-1-yl, 4-chloropyrazol-1-yl, 4-fluoropyrazol-1-yl or 3-chloropyrazol-1-yl.

8. A pesticidal composition, which comprises at least one compound of formula I according to claim 1 or, where appropriate, a tautomer thereof, in each case in free form or in agrochemically utilizable salt form, as active ingredient and at least one auxiliary.

9. A method for controlling pests, which comprises applying a composition according to claim 8 to the pests or their environment with the exception of a method for treatment of the human or animal body by surgery or therapy and diagnostic methods practised on the human or animal body.

10. A method for the protection of plant propagation material from the attack by pests, which comprises treating the propagation material or the site, where the propagation material is planted, with a composition according to claim 8.
